(19) United States
(12) Patent Application Publication
(10) Pub. No.: US 2011/0152313 A1
(43) Pub. Date: Jun. 23, 2011

(54) SYNTHESIS OF AGELADINE A AND ANALOGS THEREOF

(75) Inventors: Peter Helmuth Karuso, New South Wales (AU); Sudhir Ramnathrao Shengule, New South Wales (AU)

(73) Assignee: MACQUARIE UNIVERSITY, North Ryde, New South Wales (AU)

(21) Appl. No.: 12/999,046

(22) PCT Filed: Jun. 19, 2009
(86) PCT No.: PCT/AU2009/000794

§ 371 Date: Mar. 8, 2011

(30) Foreign Application Priority Data
Jun. 20, 2008 (AU) 2008903152

(51) Int. Cl. A61K 31/437; C07D 471/02; A61P 35/00; A61P 27/02

(52) U.S. Cl. 514/303; 546/118

(57) ABSTRACT

The invention describes a one pot process for synthesizing a compound of structure (I), or a tautomer thereof. A compound of structure (II), or a tautomer thereof, and an aldehyde of structure R^{d}CHO are condensed to form a condensation product. The resulting condensation product is then oxidized in the same reaction mixture to produce the compound of structure (I) or a tautomer thereof.

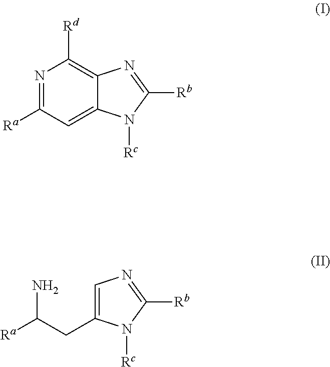

### TECHNICAL FIELD

The present invention relates to a new process for synthesis of ageleladine A and of analogs of ageladine A and the anticancer activity of these analogs.

### BACKGROUND OF THE INVENTION

Ageladine A has been isolated in small amounts from the marine sponge Agelas nakamurai (Fujita, M.; Nakao, Y.; Matsunaga, S.; Seiki, M.; Itoh, Y., et al. J. Am. Chem. Soc. 2003, 125, 15700-1). The compound is the first example of an imidazolopyridine natural product. Ageladine A has shown inhibition against various matrix metalloproteinases (MMPs) at micromolar levels and strong anti-angiogenic activity that is believed to be associated with its MMP inhibition. However, ageladine A does not function as an MMP inhibitor through zinc complexation and thus has a novel mode of action.

The important biological activity and unusually compact structure have made ageladine A a target for the synthetic chemist. To date four total syntheses have been reported: Weinreb reported the first total synthesis of ageladine A in 13 steps using a 6π-azaelectrocyclization and Suzuki-Miyaura coupling of N-Boc-pyrrole-2-boronic acid and chloropyridine as a key step (Meketa, M. L.; Weinreb, S. M. Org. Lett. 2006, 8, 1446). Soon after, Karuso reported the concise synthesis of ageladine A based on biomimetic principles in just 3 steps. Weinreb reported the third total synthesis using a variation of his original method to overcome the low yield of the last step but still employing a 6π-2-azatriene electrocyclization for the formation of the imidazolopyridine moiety but still containing 13 linear steps with an overall yield of just 4% (Meketa, M. L.; Weinreb, S. M.; Nakao, Y.; Fusetani, N. J. Org. Chem. 2007, 72, 4892-9; Meketa, M. L.; Weinreb, S. M. Org. Lett. 2007, 9, 853-5). Most recently Ando et al. reported a fourth synthesis of ageladine A, based on the Karuso synthesis, employing a Pictet-Spengler cyclization between the N-Boc-2-AH and N-protected 2-formyl pyrroles as the key step (Ando, N.; Terashima, S. Bioorg. Med. Chem. Lett. 2007, 17, 4495-9). In the Ando synthesis, dehydrogenation was effected in two steps using IBX and then MnO₂ instead of choranil, used by Karuso, which was found to improve the overall yield. The limited material available from nature as well as synthetic sources have hampered further investigations in. vivo. The syntheses of ageladine A that have so far been devised suffer from poor overall yield, lengthy synthetic schemes and/or lack of versatility to provide ready access to analogs with similar or better biological activity.

Accordingly, there is a need for an improved method for the synthesis of ageladine A and its analogs.

Angiogenesis, plays a critical role in many diseases, including hyperproliferative disorders like cancer. In cancer, tumors grow and metastasize in part by secreting angiogenic substances, such as VEGF, that can induce capillary growth into the tumor. MMP and kinase inhibition are both activities associated with inhibition of angiogenesis. Many kinases have been implicated in angiogenesis signalling. There are a number of types of new treatments in development including anti-angiogenic drugs, interferon, antibody-based technologies, and vaccines. It is not yet known which of these approaches, or combinations of approaches will be the most effective. Anti-angiogenesis drugs offer the possibility of being used alone or in combination with other classes of anticancer drugs. They have been shown to work well when given along with chemotherapy.

Within the anti-angiogenic class there are multiple ways that a drug could prevent the formation of new blood vessels, and hence the effectiveness and side effect profile of different drug candidates could vary. Several anti-angiogenesis drugs have been approved by the FDA but all have more or less severe side-effects.

Age related macular degeneration (AMD) of the eye is the most common cause of vision loss in the US and Australia in those aged 50 or older. AMD is essentially an angiogenic disease where there is abnormal growth of blood vessels in the retina. Anti-angiogenesis drugs slow the progression of ‘wet AMD’ by inhibiting blood vessel growth in the retina. The anticancer drug Avastin, is approved for use in wet AMD but there is only a small number of antibody-based drugs for the treatment of wet AMD, all of which have more or less severe side effects that must be injected into the eye on a regular basis because the drugs are not orally active.

There is thus a need to develop new drugs that can be used to treat hyperproliferative diseases and/or other diseases associated with angiogenesis such as macular degeneration. Suitable drugs would preferably be small molecule drugs. They would preferably be orally active.

### OBJECT OF THE INVENTION

It is an object of the present invention to substantially overcome or at least ameliorate one or more of the above disadvantages. It is a further object to at least partially satisfy the above need.

### SUMMARY OF THE INVENTION

The present application claims priority from Australian provisional patent application number 2008903152, the entire contents of which are incorporated herein by cross-reference.

In a first aspect of the invention there is provided a process for synthesizing a compound of structure I, or a tautomer thereof, said process comprising:
condensing a compound of structure II, or a tautomer thereof, and an aldehyde of structure R^{d}CHO to form a condensation product; and
oxidizing the condensation product to produce the compound of structure I or tautomer thereof;

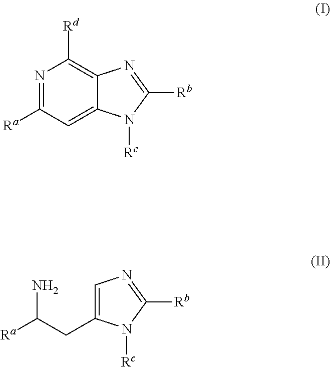

wherein:
R^{a} is R¹, COOR¹ or CONHR¹,
—R^{b} is H, —NR¹R², —OR¹, —SR¹, —SO₂R¹, or —NR³, where R³ comprises a hydrocarbon chain which, together with the nitrogen to which it is attached, forms a ring structure, R^{c} is R¹,
R^{d} is an optionally substituted aromatic or heteroaromatic group,
R¹ and R² are, independently, H, alkyl, aryl or acyl, and are, except if they are H, optionally substituted, wherein each R¹ is independently as defined above.

The process may be a one-pot process.

The following options may be used in conjunction with the first aspect, either separately or in any suitable combination.

R¹ and R² may, except if they are H, be optionally substituted with one or more hydroxy, halo or amino groups. They may be optionally substituted with one or more hydroxy, halo, amino, amide, thiol, thioether, ether, ester or nitro group, or with some other functional group.

The process may be such that the condensation product is not isolated before the step of oxidizing. It may be a “one-pot” process or synthesis. It may be a multi-step process or synthesis. It may be conducted such that no purification step is conducted between the steps of condensing and oxidizing. It may be conducted such that no isolation step is conducted between the steps of condensing and oxidizing.

The compound of structure II may be 2-aminohistamine. The aldehyde may be N-Boc-4,5-dibromopyrrole-2-carboxaldehyde. The compound of structure I may be ageladine A.

The step of oxidizing may comprise heating the crude reaction product (or the condensation product) in the presence of palladium on charcoal catalyst. The heating may be conducted without first isolating the condensation product from the reaction mixture. The heating may comprise refluxing in a solvent, for example in ethanol, or the heating may be under high pressure in a flow reactor, or it may be some other form of heating.

The step of oxidizing may comprise heating the crude reaction product (or the condensation product) in the presence of a quinone. The heating may be conducted without first isolating the condensation product from the reaction mixture. The quinone may be chloranil or DDQ (2,3-dichloro-5,6-dicyanobenzoquinone). The heating may comprise refluxing in a halogenated solvent. The halogenated product may be for example chloroform, dichloromethane, trichloroethane, dichloroethylene, chlorobenzene, tetrachloroethylene or hexachloroethane.

The step of oxidizing may comprise heating the crude reaction product (or the condensation product) in the presence of sulfur. The heating may be conducted without first isolating the condensation product from the reaction mixture. The heating may comprise refluxing in a suitable solvent or heating in a microwave reactor.

The step of oxidizing may comprise heating the product (or the condensation product) in the presence of IBX (2-iodoxybenzoic acid). The heating may be conducted without first isolating the condensation product from the reaction mixture. The heating may comprise refluxing in a suitable solvent or heating in a microwave reactor.

The step of condensing may comprise reacting the compound of structure II with the aldehyde in the presence of a base. The base may be a tertiary amine or it may be an inorganic base such as potassium hydroxide, sodium hydroxide etc. It may be an alkali metal hydroxide or it may be alkali metal carbonate or it may be some other suitable base. Optionally, molecular sieves may be added to accelerate the reaction.

The process may additionally comprise isolating the compound of structure I or tautomer thereof It may additionally comprise purifying the compound of structure I or tautomer thereof.

In an embodiment, the compound of structure I is 2-aminohistamine and the aldehyde is N-Boc-4,5-dibromopyrrole-2-carboxaldehyde, whereby the compound of structure I is ageladine A.

In some embodiments, R^{b} is NR¹R².

In other embodiments, R^{b} is XH where X is O or S, whereby the compounds of structure I and structure II exist primarily as their tautomers in which the group —N═C(R^{b})—N(H)— is replaced by —N(H)—C(═X)—N(H)—.

The invention also provides a compound of structure I, or a tautomer, pharmaceutically acceptable salt or ester thereof, said compound, tautomer, salt or ester being made by the process of the first aspect. It further provides a compound of structure I, or a tautomer, pharmaceutically acceptable salt or ester thereof, which is makeable by the process of the first aspect. In both of these options, R^{d} may be an optionally substituted heteroaromatic group. In both of these options, it is preferred that R^{a}, R^{b}, R^{c} and R^{d} are not represented by the combinations shown in Table A below:

TABLE A

| R^{a} | R^{b} | R^{c} | R^{d} |
|---|---|---|---|
| H | NH₂ | H | 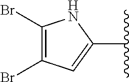 |
| H | NH₂ | H | 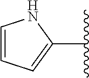 |
| H | NH₂ | H | 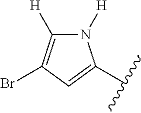 |
| H | NH₂ | H | 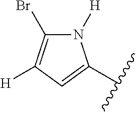 |
| H | NH₂ | H | 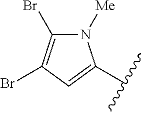 |
| H | NH₂ | H | 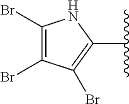 |
| H | NHMe | H | 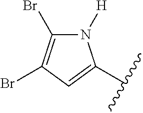 |
| H | NH₂ | Me | 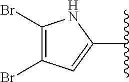 |
| H | NHMe | Me | 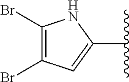 |
| H | NMe₂ | H | 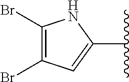 |
| H | H | H | 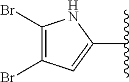 |
| H | NH₂ | Bom | 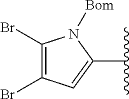 |
| H | NH₂ | Bom | 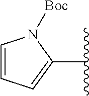 |
| H | NH₂ | Bom | 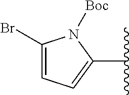 |
| H | NH₂ | Bom | 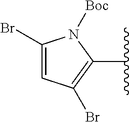 |
| H | NH₂ | H | 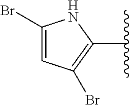 |
| H | N₃ | Bom | 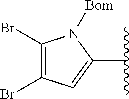 |
| H | SO₂Me | Bom | 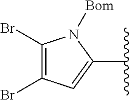 |
| H | SOMe | Bom | 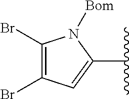 |
| H | SMe | Bom | 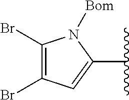 |
| H | NHBoc | H | 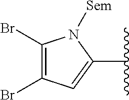 |
| H | NMeBoc | H | 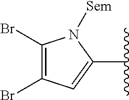 |
| H | NMeBoc | Me | 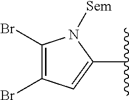 |
| H | H | H | 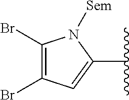 |
| H | H | Me | 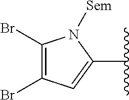 |
| H | Br | H | 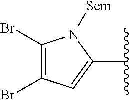 |
| H | NMe₂ | H | 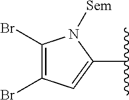 |
| H | NHMe | Me | 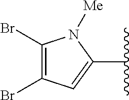 |
| CO₂Me | H | H | Ph |

In the above table (and elsewhere in this specification) Boc represents t-butyloxycarbonyl and Born represents benzyloxymethyl.

In a second aspect of the invention there is provided a composition for the treatment of a hyperproliferative disorder, said composition comprising a compound of structure I as defined above or a tautomer thereof or a pharmaceutically acceptable salt or ester thereof or a combination of any two or more of said compound or tautomer, salt and ester, together with one or more pharmaceutically acceptable carriers and/or adjuvants.

It is preferred that R^{a}, R^{b}, R^{c} and R^{d} are not represented by the combinations shown in Table A above. The compound of structure I may be such that R^{d} is an optionally substituted heteroaromatic group. The compound of structure I may be such that R^{d} is 2-pyridyl or a substituted 2-pyridyl group.

The compound of structure I, or tautomer thereof, may be made according to the first aspect.

In some embodiments, R¹ and R² are substituted with one or more hydroxy, halo or amino groups. In particular embodiments, R^{a} and R^{c} are both H, R^{b} is NH₂ and R^{d} is selected from the group consisting of furan-2-yl, thiophen-2-yl, furan-3-yl, thiophen-3-yl, pyridin-2-yl, pyridin-3-yl, quinolin-3-yl, phenyl, benzothiophen-3-yl and indol-3-yl, or wherein R^{a} is COOH, R^{b} and R^{c} are both H and R^{d}=2-thiophenyl, 2-pyridinyl or 6-bromo-2-pyridinyl, or wherein R^{a}, R^{b} and R^{c} are all H and R^{d} is 2-pyridinyl.

In a third aspect of the invention there is provided a method of treating a hyperproliferative disorder in a patient comprising administering to said patient a therapeutically effective amount of a compound of structure I as defined above, or a tautomer thereof, or a pharmaceutically acceptable salt or ester thereof or a combinations of any two or more of said compound, salt and ester, or of a composition according to the second aspect.

The compound of structure I, or tautomer thereof, may be made according to the first aspect. It is preferred that R^{a}, R^{b}, R^{c} and R^{d} are not represented by the combinations shown in Table A above. The compound of structure I may be such that R^{d} is an optionally substituted heteroaromatic group.

The hyperproliferative disorder may be a cancer.

The compound may comprise 4-(pyridin-2-yl)-1H-imidazo[4,5-c]pyridin-2-amine.

In a fourth aspect of the invention there is provided use of a compound made by the process of the first aspect, or a composition according to the second aspect, for the treatment of a hyperproliferative disorder, e.g. for the treatment of a cancer, or for an angiogenic disease, such as wet AMD. It is preferred that R^{a}, R^{b}, R^{c} and R^{d} are not represented by the combinations shown in Table A above. The compound of structure I may be such that R^{d} is an optionally substituted heteroaromatic group.

In a fifth aspect of the invention there is provided use of a compound of structure I as defined above, or a tautomer thereof, or of a compound made by the process of the first aspect, for the manufacture of a medicament for the treatment of a hyperproliferative disorder, e.g. a cancer, or of an angiogenic disease, e.g. wet AMD. It is preferred that R^{a}, R^{b}, R^{c} and R^{d} are not represented by the combinations shown in Table A above. The compound of structure I may be such that R^{d} is an optionally substituted heteroaromatic group.

In a sixth aspect of the invention there is provided a compound of structure I

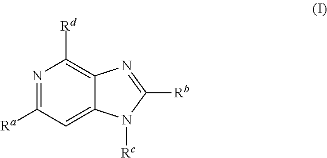

wherein:
R^{a} is R¹, COOR¹ or CONHR¹,
—R^{b} is H, —NR¹R², —OR¹, —SR¹, —SO₂R¹, or —NR³, where R³ comprises a hydrocarbon chain which, together with the nitrogen to which it is attached, forms a ring structure,
R^{c} is R¹,
R^{d} is an optionally substituted heteroaromatic group,
R¹ and R² are, independently, H, alkyl, aryl or acyl, and are, except if they are H, optionally substituted, wherein each R¹ is independently as defined above,
or a tautomer, pharmaceutically acceptable salt or ester thereof.

In the sixth aspect of the invention, R^{a}, R^{b}, R^{c} and R^{d} are not any of the combinations shown in Table A above.

In some embodiments, R¹ and R² are substituted with one or more hydroxy, halo or amino groups.

R¹ and R² may independently, except if they are H, be optionally substituted with one or more hydroxy, halo, amino, amide, thiol, thioether, ether, ester or nitro group, or with some other functional group.

Particular embodiments of the sixth aspect are such that R^{a} and R^{c} are both H, R^{b} is NH₂ and R^{d} selected from the group consisting of furan-2-yl, thiophen-2-yl, furan-3-yl, thiophen-3-yl, pyridin-2-yl, pyridin-3-yl, quinolin-3-yl, phenyl, benzothiophen-3-yl and indol-3-yl, or such that R^{a} is COOH, R^{b} and R^{c} are both H and R^{d} is thiophen-2-yl, pyridin-2-yl or 6-bromopyridin-2-yl, or such that R^{a}, R^{b} and R^{c} are all H and R^{d} is pyridin-2-yl.

The compound of structure I may be such that R^{d} is an optionally substituted heteroaromatic group.

R^{d} may be thiophen-2-yl, pyridine-2-yl or quinolin-3-yl, optionally substituted. In this case the compound may have anti-angiogenic activity. The anti-angiogenic activity may be such that at least 50% of new blood vessel growth is inhibited at a concentration of said compound of <100 μg/mL. R^{d} as pyridine-2-yl, or substituted pyridine-2-yl, are preferred.

R^{d} may be furan-2-yl, thiophen-2-yl, furan-3-yl, thiophen-3-yl, pyridin-2-yl, pyridin-3-yl, quinolin-3-yl, phenyl or indol-3-yl. In this case the compound may have kinase inhibitor activity. The kinase activity may be such that the compound has IC₅₀<100 μM.

R^{d} may be furan-2-yl, thiophen-2-yl, thiophen-3-yl, pyridin-2-yl, pyridin-3-yl, quinolin-3-yl, benzothiophen-3-yl or indol-3-yl. In this case the compound may have MMP inhibitor activity.

In a seventh aspect of the invention there is provided a pharmaceutical composition comprising a compound according to the sixth aspect, or a tautomer thereof, or a pharmaceutically acceptable salt or ester thereof or a combination of any two or more of said compounds, tautomers, salts and esters, together with one or more pharmaceutically acceptable carriers and/or adjuvants. The compound according to the sixth aspect, or tautomer, pharmaceutically acceptable salt or ester thereof, may be made according to the process of the first aspect. The composition may be for the treatment of a cancer. It may be for the treatment of age related macular degeneration. It may be for the treatment of a hyperproliferative disorder or an angiogenic disease.

In an eighth aspect of the invention there is provided a method of treating a hyperproliferative disorder or an angiogenic disease in a patient comprising administering to said patient a therapeutically effective amount of a compound according to the sixth aspect, or made by the first aspect, or a tautomer thereof, or a pharmaceutically acceptable salt or ester thereof or a combinations of any two or more of said compound, salt and ester, or of a composition according to the seventh aspect, wherein said compound is effective against said disorder or disease.

In one embodiment R^{a} and R^{c} are both H, R^{b} is NH₂ and R^{d} is selected from the group consisting of furan-2-yl, thiophen-2-yl, furan-3-yl, thiophen-3-yl, pyridin-2-yl, pyridin-3-yl, quinolin-3-yl, phenyl, benzothiophen-3-yl and indol-3-yl, or R^{a} is COOH, R^{b} and R^{c} are both H and R^{d} is thiophen-2-yl, pyridin-2-yl or 6-bromopyridin-2-yl, or such that R^{a}, R^{b} and R^{c} are all H and R^{d} is pyridin-2-yl. The method may be a method of treating a cancer. It may be a method of treating age related macular degeneration.

In a ninth aspect of the invention there is provided use of a compound according to the sixth aspect, or made by the first aspect, or a composition according to the seventh aspect, for the treatment of a hyperproliferative disorder or an angiogenic disease.

In a tenth aspect of the invention there is provided use of a compound according to sixth aspect or made by the first aspect, for the manufacture of a medicament for the treatment of a hyperproliferative disorder or of an angiogenic disease.

In the tenth aspect, the treatment may be a treatment of an angiogenic disease. It may be a treatment of age related macular degeneration.

In an eleventh aspect of the invention there is provided use of a compound according to the sixth aspect, or made by the first aspect, wherein R^{a} and R^{c} are both H, R^{b} is NH₂ and R^{d} is thiophen-2-yl, pyridine-2-yl or quinolin-3-yl, or wherein R^{a} is COOH, R^{b} and R^{c} are both H and R^{d} is thiophen-2-yl, pyridin-2-yl or 6-bromopyridin-2-yl, or such that R^{a}, R^{b} and R^{c} are all H and R^{d} is pyridin-2-yl, said compound having anti-angiogenic activity whereby at least 50% of new blood vessel growth is inhibited at a concentration of said compound of <100 μg/mL, for inhibiting angiogenesis.

In a twelfth aspect of the invention there is provided use of a compound according to the sixth aspect, or made by the first aspect, wherein R^{a} and R^{c} are both H, R^{b} is NH₂ and R^{d} is furan-2-yl, thiophen-2-yl, furan-3-yl, thiophen-3-yl, pyridin-2-yl, pyridin-3-yl, quinolin-3-yl, phenyl or indol-3-yl, said compound having kinase inhibitor activity of IC₅₀<100 μM, for inhibiting kinase activity.

In a thirteenth aspect of the invention there is provided use of a compound according to the sixth aspect, or made by the first aspect, wherein R^{a} and R^{c} are both H, R^{b} is NH₂ and R^{d} is furan-2-yl, thiophen-2-yl, thiophen-3-yl, pyridin-2-yl, pyridin-3-yl, quinolin-3-yl, benzothiophen-3-yl or indol-3-yl, said compound having MMP inhibitor activity, for inhibiting MMP activity.

### DETAILED DESCRIPTION OF THE PREFERRED EMBODIMENTS

The present invention provides a process for synthesizing a compound of structure I, or a tautomer thereof. The process may conveniently be conducted without purification and/or isolation of the condensation product that is formed initially (i.e. it may be conducted as a one-pot process), although if desired, such purification and/or isolation may be performed. It is a benefit of the present invention that no isolation of intermediate species is required. This avoids the loss of yield that commonly attends isolation and purification of intermediates, as well as reducing the time and effort required for the process. Additionally, in many embodiments of the invention, any protecting groups required on the reagents used in the synthesis are lost during the synthesis, so that no separate deprotection step is required. Thus the process may be one that has no separate deprotection steps, i.e. may be one that has no steps in which only deprotection occurs.

The process of the invention may be conducted, at least in part, in a water compatible or water miscible solvent. It may be conducted in an aqueous solvent. The aqueous solvent may comprise water. It may additionally comprise a second solvent which is miscible with water. The second solvent may be an alcohol. It may be a C1 to C4 alcohol, such as methanol, ethanol, propanol, isopropanol, tert-butanol etc. It may be a diol, e.g. ethane-1,2-diol or propane-1,3-diol. It may be some other type of water miscible solvent.

A scheme for ageladine A synthesis, using the process of the present invention, is shown in Scheme 1 below.

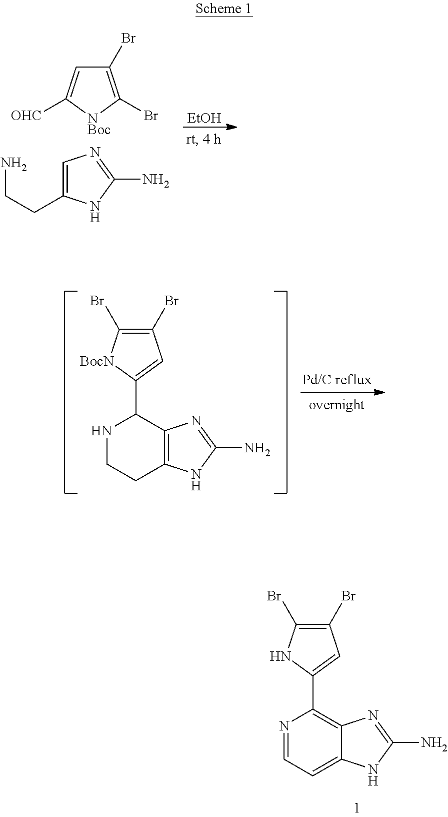

A more generalized process for making ageladine A and analogs thereof is provided below in Scheme 2.

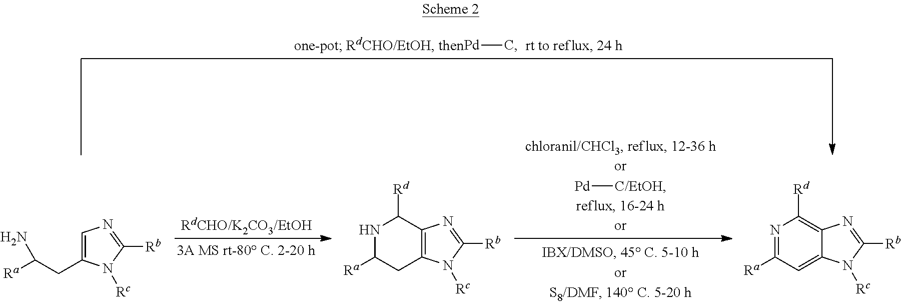

The first step of the process comprises condensing compound of structure II, or a tautomer thereof, and an aldehyde of structure R^{d}CHO to form a condensation product. The initial condensation product has structure III, shown below:

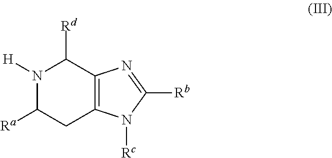

The various substituents in structure III are as defined for structures I and II earlier. In general, compound III is not isolated or purified in any way prior to being oxidized to compound I, although some of the solvent may in some cases be removed. The term “one-pot” may be taken to mean that the initial condensation product is not isolated or purified in any way prior to being oxidized to compound I. The reaction mixture following at least partial conversion of I to III (which therefore comprises compound III) may be physically transferred to another vessel if required, and this is envisaged within the term “one pot” provided that compound III is not isolated or purified in any way prior to being oxidized to compound I.

The aldehyde may be used in a slight molar excess over the compound of structure II (or tautomer thereof) or it may be used in a molar equivalent amount. The molar excess of aldehyde over the compound of structure II (or tautomer thereof) may be about 0 to about 50%, or about 0 to 30, 0 to 20, 0 to 10, 0 to 5, 5 to 50, 10 to 50, 20 to 50, 10 to 30 or 10 to 20%, e.g. about 0, 5, 10, 15, 20, 25, 30, 35, 40, 45 or 50%.

The condensation step is commonly conducted in a solvent. Suitable solvents include alcohols, such as ethanol, methanol, isopropanol etc. The solvent may be capable of dissolving the compound of structure I. It may be capable of dissolving the condensation product. The condensation step may be conducted in the presence of a base. Suitable bases include amines, preferably tertiary amines, more preferably tertiary amines which are soluble in the solvent. Examples include triethylamine, pyridine, N,N-dimethylaniline etc. The base may be an inorganic base. Suitable inorganic bases include alkali metal hydroxide and carbonates or mixtures of these. Examples include lithium hydroxide, sodium hydroxide, potassium hydroxide, sodium carbonate, potassium carbonate and cesium carbonate. The base may be used in at least 1 mole equivalent relative to either of the reagents, or from 1 to 2 mole equivalents, or about 1 to 1.5, 1 to 1.2, 1.2 to 1.5 or 1.5 to 2 mole equivalents, e.g. about 1, 1.1, 1.2, 1.3, 1.4, 1.5, 1.6, 1.7, 1.8, 1.9 or 2 mole equivalents. The condensation may be conducted in the absence of added base (other than the reagents for the reaction). It may be conducted in the absence of catalyst. It may be conducted in the presence of a Lewis acid catalyst. Suitable catalysts include transition metal and lanthanide triflates, and mixtures of these. Examples include scandium triflate, lanthanum triflate, samarium triflate, indium triflate and ytterbium triflate. The catalyst may be added in 0.01 to 0.2 mole equivalents relative to either one of the reagents, or about 0.01 to 0.1, 0.01 to 0.05, 0.1 to 0.2, 0.05 to 0.15 or 0.08 to 0.15 mole equivalents, e.g. about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19 or 0.2 mole equivalents. The reaction may also be conducted in the presence of molecular sieves. Suitable molecular sieves may be 3 Å, 4 Å or 5 Å pore size. These may be ground to a fine powder and/or activated in an oven or microwave oven before use. The reaction is commonly conducted at room temperature, but may conveniently be conducted at between 10 and 80° C., or about 10 to 50, 10 to 40, 10 to 30, 10 to 20, 20 to 50, 50 to 80, 30 to 60, 15 to 70, 25 to 70, 25 to 50 or 15 to 25 ° C., e.g. about 10, 15, 20, 25, 30, 35, 40, 45, 50, 55, 60, 65, 70, 75 or 80° C., or at some other temperature. Alternatively, the reaction may be conducted in a microwave reactor at the same temperatures. The time for the reaction will depend in part on the temperature used. The time is commonly about 3 to 6 hours, but may be about 1 to about 10 hours, or about 1 to 5, 1 to 3, 2 to 10, 5 to 20, 2 to 6 or 3 to 5 hours, e.g. about 1, 2, 3, 4, 5, 6, 7, 8, 9, 10, 11, 12, 13, 14, 15, 16, 17, 18, 19 or 20 hours. The reaction mixture may be agitated, e.g. stirred or shaken, during the reaction. The various reagents and catalyst (if present) may be added in any desired order. Commonly the compound of structure II (or a tautomer or salt thereof) will be dissolved in the solvent. The aldehyde will then be added, followed, if used, by the catalyst or base.

The second step of the process comprises oxidizing the condensation product to produce the compound of structure I or tautomer thereof. As noted previously, this step may be conducted without separation or purification of the condensation product. Thus the reaction mixture from the previous (condensation) step may be used for the oxidation step. There may be no removal of solvent from the reaction mixture from the previous step. Thus following the stirring of the reaction mixture for the requisite time (as described above) the oxidation step may be conducted. Alternatively, the solvent may be at least partially removed from the crude reaction mixture to provide a crude product, which may then be used without purification in the subsequent (oxidation) step. As a further alternative the condensation product may be purified (optionally following at least partial removal of the solvent), e.g. by column chromatography or flash chromatography or HPLC or some other suitable method prior to the oxidation step.

The step of oxidizing may comprise adding an oxidant to the condensation product. The oxidant may comprise a quinone. It may comprise chloranil. It may comprise 2-iodoxybenzoic acid (IBX). It may comprise sulfur (S₈). The oxidation may comprise adding an oxidizing catalyst. The oxidizing catalyst may comprise an iron-group metal (iron, cobalt, nickel, ruthenium, rhodium, palladium, osmium, iridium or platinum) or an oxide of an iron-group metal, said metal or oxide being optionally disposed on a suitable support (e.g. carbon). Examples include, but are not limited to, palladium metal (e.g. 5% or 10% Pd on carbon), platinum oxide or Raney nickel. Commonly the oxidation step is conducted at elevated temperature, for example at reflux. It may be conducted at about 40 to about 150° C., or about 50 to 150, 40 to 100, 50 to 100, 100 to 150, 40 to 80, 70 to 100 or 60 to 80° C., e.g. about 40, 50, 60, 70, 80, 90, 100, 110, 120, 130, 140 or 150° C., or may be conducted at some other temperature. It may be conducted at a pressure of about 1-100 atmospheres using a flow reactor. The pressure may be about 1 to 50, 1 to 20, 1 to 10, 1 to 5, 1 to 2, 2 to 100, 5 to 100, 10 to 100, 20 to 100, 50 to 100, 5 to 50, 5 to 20 or 10 to 50 atmospheres, e.g. about 1, 2, 3, 4, 5, 6, 7, 8, 9, 10, 15, 20, 25, 30, 35, 40, 45, 50, 60, 70, 80, 90 or 100 atmospheres. Optionally a hydrogen acceptor such as cyclopentene, cyclohexene or decalene may be added. Suitable solvents for this step include alcohols, such as methanol, ethanol, propanol or butanol when using an iron-group metal as oxidizing catalyst. The reaction may be conducted at the boiling point of the solvent used. Halogenated solvents such as chloroform, dichloromethane, trichloroethane, dichloroethylene, chlorobenzene, tetrachloroethylene or hexachloroethane may be particularly suitable when using a quinone as oxidant.

Other options for the step of oxidizing are well known. These are described for example in “Advanced Organic Chemistry; Reactions, Mechanisms and Structure”, 4th Edition, J. March, John Wiley and Sons, Inc. 1992 (for example in Chapter 9: Oxidations and Reductions) and in “Comprehensive Organic Transformations: A Guide to Functional Group Preparations”, R. C. Larock, VCH Publishers Inc. 1989 (for example in Chapter 5: Aromatization), the contents of which are incorporated herein by cross-reference.

In the event that a non-catalytic oxidant is used, it may be used in molar excess over the condensation product or may be used in a molar equivalence. The molar excess may be about 0 to about 200% (i.e. 1 to 3 moles of oxidant per mole of condensation product), or about 0 to 100, 0 to 50, 0 to 20, 20 to 200, 50 to 200, 100 to 200, 50 to 150 or 80 to 120%, e.g. about 0, 10, 20, 30, 40, 50, 60, 70, 80, 90, 100, 110, 120, 130, 140, 150, 160, 170, 180, 190 or 200%. In the event that an oxidizing catalyst is used, it may be used at about 100 to about 500 mg per mole of condensation product, or about 100 to 300, 200 to 500 or 200 to 300 mg/mol, e.g. about 100, 150, 200, 250, 300, 350, 400, 450 or 500 mg per mole of condensation product.

The oxidation may lead to formation of an aromatic structure. It may be an aromatization. It may be a dehydrogenation.

In some cases one or both of the reagents (i.e. the aldehyde and the compound of structure II or a tautomer or salt thereof) may have a protecting group. In this instance, the process may comprise deprotecting, i.e. removing the protecting group, after the step of oxidizing in order to produce the compound of structure I. Alternatively, the deprotection (removal of the protecting group) may be effected during the step of oxidizing (optionally using the oxidizing reagent or catalyst) and no separate deprotection step may be required.

The final product may be purified by standard procedures. These include column chromatography, preparative hplc, preparative gc, flash chromatography, crystallization, sublimation or a combination of any two or more of these.

In structures I to III, the following options are used:

Alkyl—may be straight chain, branched chain or cyclic alkyl groups, and may comprise more than one of these (e.g. cyclohexylmethyl). They may be C1 to C12, C1 to C6, C1 to C3, C2 to C12, C6 to C12 or C2 to C6, e.g. may have 1, 2, 3, 4, 5, 6, 7, 8, 9, 10, 11 or 12 carbon atoms, or may have more than 12 carbon atoms. Examples include methyl, ethyl, propyl, isopropyl, butyl, isobutyl, t-butyl, pentyl, neopentyl, cyclopentyl, hexyl, cyclohexyl etc. They may optionally be substituted, e.g. with hydroxyl, halogen, amine, amide, thiol, thioether, hydroxyl, ether, ester or nitro group, or with some other functional group or with an aromatic or heteroaromatic group (see below).

Aromatic—may be monocyclic (e.g. phenyl), fused (e.g. naphthyl, anthracyl) or linked (e.g. biphenyl, terphenyl). They may optionally be substituted with an aromatic, heteroaromatic or alkyl group or more than one of these (e.g. may be tolyl, phenylanthracyl etc.). They may have 6 to 20 carbon atoms in the ring structure, or 6 to 12, 8 to 20 or 8 to 16, e.g. 6, 8, 10, 12, 14, 16, 18 or 20 carbon atoms. They may optionally be substituted, e.g. with hydroxyl, halogen, amine, amide, thiol, thioether, hydroxyl, ether, ester or nitro group, or with some other functional group.

Heteroaromatic—may be monocyclic (e.g. pyridyl), fused (e.g. quinolinyl) or linked (e.g. bipyridyl). They may have 1, 2, 3 or more than 3 heteroatoms. Each heteroatom may independently be N, S or O. The heteroaromatic group may optionally be substituted, with an alkyl, aromatic or heteroaromatic group or more than one of these. They may optionally be substituted, e.g. with hydroxyl, halogen, amine, amide, thiol, thioether, hydroxyl, ether, ester or nitro group, or with some other functional group.

In the event that R^{b} is NR¹R², and neither R¹ nor R² is H, R¹ and R² may, together with the nitrogen atom to which they are attached, form a cyclic structure. The cyclic structure may have between about 4 and about 8 atoms in the ring, e.g. 4, 5, 6, 7 or 8 atoms.

The present specification discloses a one-pot synthesis of ageladine A based on a Pictet-Spengler reaction between 2-aminohistamine and N-Boc-4,5-dibromopyrrole-2-carboxaldehyde. It also discloses a method for readily synthesizing analogs of ageladine A, based on the condensation of 4-(ω-aminoalkyl)-imidazoles with aldehydes in one pot.

Referring to Scheme 1, 2-aminohistamine (2-AH) is a biogenetic amine related to the amino acid histidine by decarboxylation and amination. This compound is a precursor to many natural products. The other half of the molecule can be envisaged arising from proline via dehydrogenation, bromination (haloperoxidase) and reduction of the carboxylic acid to 4,5-dibromo-2-pyrrole carboxaldehye. This compound, protected as the N-Boc or N-ethyl-2-phenylsulfone derivative is mixed with 2-AH in ethanol for six hours, followed by the addition of palladium on charcoal. The reaction is refluxed overnight to afford ageladine A after chromatography on silica.

Referring to Scheme 2, analogs of ageladine A may be synthesized in two steps from 2-AH. Thus, in an example of a suitable process, to a stirred solution of 2-AH in ethanol is added triethylamine and an aldehyde. The reaction mixture is stirred at room temperature for 6 h, after which the solvent is removed under reduced pressure and the reaction mixture then further subjected to flash chromatography over silica gel using a gradient of 5:95-30:70 (MeOH:DCM saturated with ammonia) yielding the tetrahydro-intermediate (III) (70-99%). Alternatively, the tetrahydro-intermediate (III) may be made by substituting triethylamine for potassium carbonate and 3 Å molecular sieves and refluxing the mixture for 1-2 days. To a stirred solution of the tetrahydro-intermediate (III) in chloroform was added chloranil and the reaction mixture refluxed for 20 h. Solvent was removed on a rotary evaporator and the residue obtained purified by flash chromatography over silica gel with a gradient of 5:95-15:85 (MeOH:DCM saturated with ammonia) giving the ageladine A analog (50-80%). Alternatively, dehydrogenation may be effected by heating the tetrahydro-intermediate (III) in DMSO in the presence of 1.5 equivalents of iodoxybenzene (IBX) to 45° C. for 6 hours. The solvent may be removed by freeze-drying and the lyopholized powder purified by reverse phase HPLC (0-70% acetonitrile in water containing 0.1% trifluoroacetic acid) to yield the ageladine A analog as its TFA salt (20-90%).

In the event that the aldehyde used in the synthesis contains an NH group, this may conveniently be protected, using known protecting groups. Suitable protecting groups include Boc (butyloxycarbonyl), Cbz (carbobenzyloxy), acetate, ethyl-2-phenylsulfone, or other derivative. This may also serve to increase the solubility of the reagent and thus increase yields and reduce the reaction time.

The dehydrogentation reaction may be effected with palladium metal, supported on activated carbon. A compound capable of recycling the catayst by easily being reduced and thus accepting hydrogen such as cyclopentene, cyclohexene or decalene may be optionally added. This is particularly effective for aldehydes that contain pyridine, quinoline or other nitrogen heterocycles. Another convenient method is to use IBX in DMSO at 45° C. Alternatively the dehydrogentation reaction may be effected with sulfur (S₈) in DMF. Other dehydrogenation methods may suggest themselves to one skilled in the art.

2-AH may be replaced by other amino-ethylimidazoles as described elsewhere herein. The 2-AH or analog thereof may be substituted at the 2′-position (R^{a}) to yield 6-substituted 1H-imidazo[4,5-c]pyridines.

The invention also provides a method for treating tumors, cancers, neoplastic tissue and other premalignant and normeoplastic hyperproliferative or hyperplastic disorders. The method comprises the use of ageladine analogs and derivatives thereof or pharmaceutically acceptable salts or esters thereof, as an antitumor agent by inhibiting the growth of tumors, cancers, neoplastic tissue and other premalignant and normeoplastic hyperproliferative or hyperplastic disorders. The method may be used to inhibit angiogenic mechanisms in the target cells which are generally hyperproliferative cells including tumors, cancers and neoplastic tissue along with premalignant and non-neoplastic or non-malignant hyperproliferative disorders as well as macular degeneration. Examples of tumors, cancers and neoplastic tissue that can be treated by the present method include, but are not limited to, malignant disorders such as breast cancers, osteosarcomas, angiosarcomas, fibrosarcomas and other sarcomas, lymphomas, sinus tumors, ovarian, uretal, bladder, prostate and other genitourinary cancers, colon, esophageal and stomach cancers and other gastrointestinal cancers, lung cancer, pancreatic cancers, liver cancers, kidney cancers, endocrine cancers, skin cancers, and brain or central and peripheral nervous system tumors, malignant or benign, including gliomas and neuroblastomas. Examples of pre-malignant and non-malignant hyperproliferative disorders include but are not limited to cervical carcinomas, familial intestinal polyposes such as Gardner's syndrome, oral leukoplakias, histiocytosis, keloids, hemangiomas, hyperproliferative arterial stenosis, inflammatory arthritis, hyperkeratosis and papulosquamous eruptions including arthritis. Also included are viral induced hyperproliferative diseases such as warts and EBV induced disease such as infectious mononucleosis, scar formation and the like. Examples of macular degeneration include, but are not limited to wet age related macular degeneration (wet AMD). The method may be employed with any subject known or suspected of carrying or at risk of developing a hyperproliferative disorder.

In the present specification, “treatment” of a hyperproliferative disorder refers to inhibiting or slowing the growth of hyperproliferative cell numbers as well as reducing the size of a hyperproliferative growth or a reduction in vascularization. Treatment is not necessarily a cure or complete removal of the hyperproliferative growths. A treatment effective amount is an amount effective to result in the killing, the slowing of the rate of growth of hyperproliferative cells the decrease in the size of a body of hyperproliferative cells, and or the reduction in number of hyperproliferative cells.

The active compounds may be formulated for administration in a single pharmaceutical carrier or in separate pharmaceutical carriers for the treatment of a variety of conditions. The carrier must be compatible with any other ingredients in the formulation and must not be deleterious to the patient. The carrier may be a solid or liquid or both and is preferably formulated with the compound as a unit dose formulation, such as a tablet which may contain 5% to 85% by weight of the active compound. One or more active compounds may be incorporated into the formulation, which may be prepared by any of the known techniques of pharmacy consisting essentially of admixing the components and optionally including one or more accessory ingredients.

The formulations of the present invention are those suitable for oral, rectal, buccal (e.g., sub-lingual), vaginal, parenteral (e.g., subcutasneous, intramuscular, intradermal, or intravenous), topical (both skin and mucosal surfaces, including airway surfaces) and transdermal administration, although the most suitable route in any given case will depend on the nature and severity of the condition being treated and on the nature of the particular active analog being used.

Formulations suitable for oral administration may be presented in discrete units such as capsules cachets, lozenges, or tablets each containing a predetermined amount of the active analog(s), as a powder or granules, as a solution or a suspension in an aqueous or non-aqueous liquid, or as an oil-in-water or water-in-oil emulsion or a liposomal formulation. Such formulations may be prepared by any suitable method of pharmacy which includes the step of bringing into association the active compound and a suitable carrier (which may contain one or more accessory ingredients). In general, formulations are prepared by uniformly and intimately admixing the active compound with a liquid or finely divided solid carrier, or both, and then if necessary shaping the resulting mixture. For example a tablet may be prepared by compressing or molding a powder or granules containing the active analog(s), optionally with one or more accessory ingredients. Other delivery formulations may suggest themselves to one skilled in the art.

The therapeutically effective dosages of any one active ingredient will vary somewhat from compound to compound, patient to patient, and will depend upon factors such as the condition of the patient and the route of delivery. Such dosages can be determined in accordance with known pharmacological procedures in light of the disclosure herein.

### Abbreviations

2-aminohistamine (2-AH), tert-butoxycarbonyl- (Boc-), carbobenzyloxy- (Cbz-), dimethylsulfoxide (DMSO), dichloromethane (DCM), methanol (MeOH), nuclear magnetic resonance spectroscopy (NMR), Ultraviolet-Visible spectroscopy (UV-Vis), mass spectrometry (MS), high resolution mass spectrometry (HRMS), trifluroacetic acid (TFA), room temperature (rt), fetal calf serum (FCS), Vascular endothelial growth factor (VEGF), 2-iodoxybenzoic acid (IBX), High Performance/Pressure Liquid Chromatography (HPLC).

### General Chemical Procedures

All chemical starting materials were obtained from commercial sources or synthesized according to the prior art. Final products were purified by HPLC on a Waters 600E multi solvent delivery system equipped with a 490 programmable multi-wavelength detector using a Phenomenex reverse phase HPLC column (Gemini 5 μm; 250×10 mm). LC-MS analyses were conducted on a Shimadzu LCMS-2010 EV equipped with a photodiode array detector and electrospray source. A Phenomenex reverse phase HPLC column (Gemini, 5 μm; 150×2 mm) was used for LC-MS using water/acetonitrile/0.1% formic acid as mobile phase. UV-Vis spectra were obtained from the LC-MS photodiode array detector. IR spectra were recorded on a Paragon PE1000 FTIR spectrophotometer (Perkin Elmer, USA) as KBr discs. High-resolution mass spectra were measured on a Bruker Apex 4.7 T FTICR-MS instrument. NMR spectra were recorded in 5 mm pyrex NMR tubes (Wilmald, USA; 507-PP) on Bruker DPX400 or DRX600 NMR spectrometers, operating at 400 and 600 MHz respectively for protons. Chemical shifts were referenced to the solvent peaks: δH 3.31 and δC 49.1 for CD₃OD, δH 7.25 and δC 77.01 for CDCl3 and δH 2.49 and δC 39.5 for d₆-DMSO.

### General Biological Procedures

Buffer A: 10 mM MgCl₂, 1 mM EGTA, 1 mM DTT, 25 mM Tris-HCl pH 7.5, 50 μg heparin/mL. Buffer C: 60 mM β-glycerophosphate, 15 mM p-nitrophenylphosphate, 25 mM Mops (pH 7.2), 5 mM EGTA, 15 mM MgCl₂, 1 mM DTT, 1 mM sodium vanadate, 1 mM phenyl phosphate.

### EXAMPLES

The following examples are provided to simply exemplify certain particular features of working embodiments of the present invention. The scope of the present invention should not be limited to those features exemplified.

### Example 1

This example describes the synthesis of tetrahydroageladine A and ageladine A, (I where R^{b}═NH₂, R^{d}═4,5-dibromo-2-pyrrole, R^{a}═R^{c}═H), in two steps. To a stirred solution of 2-aminohistamine (10 mg, 0.079 mmol.) in ethanol (5 mL) was added N-Boc-4,5-dibromo-2-formylpyrrole (27.9 mg, 0.079 mmol.) and scandium triflate (3.8 mg, 0.0079 mmol.) and the reaction mixture stirred at room temperature for 5 h. The solvent was then removed in vacuo leaving a brown residue, which was purified by column chromatography on silica gel using a gradient of 5:95-15:85 (MeOH:DCM saturated with ammonia) to give tetrahydroageladine A as a yellow solid (16 mg, 44%). UV-Vis (acetonitrile/water) λₘₐₓ 220 nm; ¹HNMR (400 MHz, d₆-DMSO) δ 11.61 (bs, 1H), 10.18 (bs, 1H), 5.97 (bs, 0.5H), 5.78 (bs, 0.5H), 5.52 (bs, 0.5H), 5.06 (bs, 2H), 4.11 (bs, 0.5H), 3.97 (bs, 1H), 3.06 (m, 1H), 2.5 (m, 1H), 2.33 (bm, 1H), 1.42 (s, 9H); ¹³C NMR (100 MHz, d₆-DMSO) δ 153.8, 149.2, 134.1, 125.0, 120.8, 109.6, 98.6, 96.5, 79.2, 49.1, 48.3, 39.1, 28.1, 22.0; m/z 360 (48%), 362 (100) 364 (52); HRMS found [M+Na⁺] 483.9777, C₁₅H₁₉N₅O₂⁷⁹Br⁸¹BrNa requires 483.9777. To a stirred solution of tetrahydroageladine A (10 mg, 0.0216 mmol.) in chloroform (5 mL) was added chloranil (15.9 mg, 0.065 mmol.) and the reaction mixture was heated at 80° C. (reflux) for 8 h. The solvent was removed in vacuo and the yellow residue subjected to column chromatography over silica using a gradient of 5:95-15:85 (MeOH:DCM saturated with ammonia) to yield pure ageladine A as a yellow solid (5 mg, 65%). Ageladine A was characterized as its TFA salt, by adding a drop of TFA, for comparison to the original natural product. UV (acetonitrile/water) λₘₐₓ 210, 220 (sh), 248, 275 (sh), 290, 363, 380 (sh) nm; ¹H NMR (400 MHz, MeOD) δ 8.06 (d, J=6.4 Hz, 1H), 7.42 (d, J=6.4 Hz, 1H), 7.17 (s, 1H); ¹³C NMR (100 MHz, MeOD) δ 160.8, 147.2, 136.7, 133.0, 128.5, 125.6, 115.1, 107.8, 105.4, 102.3; m/z 356 (53%), 358 (100) 360 (48).

### Example 2

This example describes the synthesis of ageladine A in a one-pot reaction. A mixture of 2-aminohistamine (0.11 mmol) and N-Boc-4,5-dibromo-2-pyrrole carboxaldehyde (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure ageladine A (6 mg; 16%) as a yellow solid, identical to material synthesized using the method of Example 1 included herein.

### Example 3

This example describes the two step synthesis of didebromoageladine A, (I where R^{b}═NH₂, R^{d}=2-pyrrole, R^{a}═R^{c}═H), in two steps. To a stirred solution of 2-AH (0.11 mmol) in ethanol (5 mL) was added triethylamine (0.16 mmol) and N-Boc-2-pyrrole carboxaldehyde (0.13 mmol). The reaction mixture was stirred at rt for 6 h, after which the solvent was removed under reduced pressure and the reaction mixture was further subjected to flash chromatography over silica gel using a gradient of 5:95-30:70 (MeOH:DCM saturated with ammonia) yielding N′-tert-Butyl-2-(2-amino-4,5,6,7-tetrahydro-1H-imidazo[4,5-c]pyridine-4-yl)1H-pyrrole-1-carboxylate (21.7 mg; 65%) as a white solid. ¹H NMR (400 MHz, CDCl₃) δ 7.15 (dd, J=3.3, 1.8 Hz, 1H), 5.99 (t, J=3.3 Hz, 1H), 5.82 (dd, J=2.6, 1.8 Hz, 1H), 5.3 (s, 1H), 4.76 (bs, 2H), 3.01-2.80 (m, 2H), 2.54-2.28 (m, 2H), 1.58 (s, 9H); ¹³C NMR (100 MHz, CDCl₃) δ 149.6, 148.1, 135.3, 125.8, 124.2, 121.9, 115.2, 109.9, 84.1, 49.0, 38.8, 28.0, 23.2; ir (KBr) 3345, 2978, 1736, 1622, 1478, 1331, 1158, 1119, 1061, 845, 729 cm⁻¹; HRMS found [M+H]⁺ 304.1781, C₁₅H₂₂N₅O₂ requires 304.1774. To a stirred solution of this tetrahydro-intermediate (III) (0.15 mmol) in chloroform (10 mL) was added chloranil (0.30 mmol) and the reaction mixture refluxed for 20 h. Solvent was removed on a rotary evaporator and the residue obtained further purified by flash chromatography over silica gel with a gradient of 5:95-15:85 (MeOH:DCM saturated with ammonia) giving pure didebromoageladine A (16 mg; 54%) as a white solid. ¹H NMR (400 MHz, CD₃OD) δ 7.93 (d, J=6.5 Hz, 1H), 7.34 (d, J=6.5 Hz, 1H), 7.23 (dd, J=2.6, 1.4 Hz, 1H), 7.18 (dd, J=4.0, 1.4 Hz, 1H), 6.42 (dd, J=3.9, 2.6 Hz, 1H); ¹³C NMR (100 MHz, CD₃OD) δ 160.2, 146.1, 136.4, 131.6, 131.5, 125.5, 123.4, 112.5, 111.8, 104.2; HRMS found [M+H]⁺ 200.0932, C₁₀H₁₀N₅ requires 200.0936.

### Example 4

This example describes the synthesis of didebromoageladine A, (I where R^{b}═NH₂, R^{d}=2-pyrrole, R^{a}═R^{c}═H), in a one-pot reaction. A mixture of 2-aminohistamine (0.11 mmol) and N-Boc-2-pyrrole carboxaldehyde (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure didebromoageladine A (7.2 mg; 33%) as a white solid. ¹H NMR (400 MHz, CD₃OD) δ 7.93 (d, J=6.5 Hz, 1H), 7.34 (d, J=6.5 Hz, 1H), 7.23 (dd, J=2.6, 1.4 Hz, 1H), 7.18 (dd, J=4.0, 1.4 Hz, 1H), 6.42 (dd, J=3.9, 2.6 Hz, 1H)

### Example 5

This example describes the two step synthesis of 4-(Furan-2-yl)-1H-imidazo[4,5-c]pyridin-2-amine, (I where R^{b}═NH₂, R^{d}=2-furan, R^{a}═R^{c}═H), in two steps. To a stirred solution of 2-Boc-2-aminohistamine (0.11 mmol) in ethanol (5 mL) was added triethylamine (0.16 mmol) and 2-furfural (0.13 mmol). The reaction mixture was stirred at rt for 6 h, after which the solvent was removed under reduced pressure and the reaction mixture was further subjected to flash chromatography over silica gel using a gradient of 5:95-30:70 (MeOH:DCM saturated with ammonia) yielding N″-tert-butyl-4-(furan-2-yl)-4,5,6,7-tetrahydro-1H-imidazo[4,5-c]pyridine-2-ylcarbamate. (30.7 mg; 92%) as a white solid. ¹H NMR (400 MHz, CDCl₃) δ 7.36 (dd, J=1.7, 0.8 Hz, 1H), 6.26 (dd, J=3.1, 1.8 Hz, 1H), 5.89-5.87 (m, 1H), 5.22 (s, 1H), 2.97 (ddd, J=13.0, 6.0, 1.7 Hz, 1H), 2.84 (ddd, J=13.0, 10.7, 4.6 Hz, 1H), 2.60-2.49 (m, 1H), 2.34 (ddd, J=16.2, 4.5, 1.2 Hz, 1H), 2.24 (bs, 1H), 2.23 (bs, 1H), 1.29 (s, 9H); ¹³C NMR (100 MHz, MeOD) δ 155.3, 150.8, 149.9, 141.4, 134.8, 116.2, 110.2, 108.0, 85.2, 50.3, 38.3, 27.6, 25.5; ir (KBr) 3443, 1724, 1647, 1389, 1350, 1145, 1110, 837, 729 cm⁻¹; HRMS found [M+H]⁺ 305.1612, C₁₅H₂₁N₄O₃ requires 305.1614. To a stirred solution of this tetrahydro-intermediate (III) (0.15 mmol) in chloroform (10 mL) was added chloranil (0.30 mmol) and the reaction mixture refluxed for 20 h. Solvent was removed on a rotary evaporator and the residue obtained further purified by flash chromatography over silica gel with a gradient of 5:95-15:85 (MeOH:DCM saturated with ammonia) giving pure 4-(Furan-2-yl)-1H-imidazo[4,5-c]pyridin-2-amine (18.6 mg; 62%) as a white solid. ¹H NMR (400 MHz, CD₃OD) δ 8.10 (d, J=6.4 Hz, 1H), 7.92 (dd, J=1.8, 0.7 Hz, 1H), 7.67 (dd, J=3.6, 0.6 Hz, 1H), 7.44 (d, J=6.4 Hz, 1H), 6.80 (dd, J=3.5, 1.8 Hz, 1H); ¹³C NMR (100 MHz, CD₃OD) δ 161.8, 150.3, 147.5, 145.4, 133.9, 133.4, 127.1, 116.9, 114.3, 107.1; HRMS found [M+H]⁺201.0777, C₁₀H₉N₄O requires 201.0776.

### Example 6

This example describes the synthesis of 4-(Furan-2-yl)-1H-imidazo[4,5-c]pyridin-2-amine, (I where R^{b}═NH₂, R^{d}═2-furan, R^{a}═R^{c}═H), in a one-pot reaction. A mixture of 2-aminohistamine (0.11 mmol) and 2-furfural (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure 4-(Furan-2-yl)-1H-imidazo[4,5-c]pyridin-2-amine (16 mg; 73%) as a white solid. ¹H NMR (400 MHz, CD₃OD) δ 8.10 (d, J=6.4 Hz, 1H), 7.92 (dd, J=1.8, 0.7 Hz, 1H), 7.67 (dd, J=3.6, 0.6 Hz, 1H), 7.44 (d, J=6.4 Hz, 1H), 6.80 (dd, J=3.5, 1.8 Hz, 1H).

### Example 7

This example describes the two step synthesis of 4-(thiophen-2-yl)-1H-imidazo[4,5-c]pyridin-2-amine, (I where R^{b}═NH₂, R^{d}=2-thiophene, R^{a}═R^{c}═H), in two steps. To a stirred solution of 2-Boc-2-AH (0.11 mmol) in ethanol (5 mL) was added triethylamine (0.16 mmol) and 2-thiofurfural (0.13 mmol). The reaction mixture was stirred at rt for 6 h, after which the solvent was removed under reduced pressure and the reaction mixture was further subjected to flash chromatography over silica gel using a gradient of 5:95-30:70 (MeOH:DCM saturated with ammonia) yielding N″-tert-butyl-4-(thiophen-2-yl)-4,5,6,7-tetrahydro-1H-imidazo[4,5-c]pyridine-2-ylcarbamate. (26 mg; 74%) as a white solid. ¹H NMR (400 MHz, CDCl₃) δ 7.20 (dd, J=5.0, 1.2 Hz, 1H), 6.89 (dd, J=5.0, 3.5 Hz, 1H), 6.70-6.68(m, 1H), 5.80 (bs, 1H), 5.40 (s, 1H), 3.00 (ddd, J=13.3, 6.2, 2.1 Hz, 1H), 2.91 (ddd, J=13.3, 10.2; 4.8 Hz, 1H), 2.56 (dddd, J=16.4, 10.1, 6.3, 1.9 Hz, 1H), 2.30 (dddd, J=16.4, 4.8, 2.1, 0.6 Hz, 1H), 2.23 (bs, 1H), 1.29 (s, 9H); ¹³C NMR (100 MHz, CDCl₃) δ 150.6, 149.9, 147.6, 134.5, 126.2, 125.7, 124.5, 118.5, 85.4, 51.8, 38.4, 27.5, 25.6; ir (KBr) 3470, 2929, 1714, 1647, 1388, 1346, 1146, 1102, 838, 809, 729 cm⁻¹; HRMS found [M+H]⁺ 321.1393, C₁₅H₂₁N₄O₂S requires 321.1385. To a stirred solution of this tetrahydro-intermediate (III) (0.15 mmol) in chloroform (10 mL) was added chloranil (0.30 mmol) and the reaction mixture refluxed for 20 h. Solvent was removed on a rotary evaporator and the residue obtained further purified by flash chromatography over silica gel with a gradient of 5:95-15:85 (MeOH:DCM saturated with ammonia) giving pure 4-(thiophen-2-yl)-1H-imidazo[4,5-c]pyridin-2-amine (22.7 mg; 70%) as a white solid. ¹H NMR (400 MHz, CD₃OD) δ 8.10 (d, J=6.4 Hz, 1H), 8.09 (dd, J=3.8, 1.1 Hz, 1H), 7.88 (dd, J=5.0, 1.1 Hz, 1H), 7.48 (d, J=6.4 Hz, 1H), 7.32 (dd, J=5.0, 3.8 Hz, 1H); ¹³C NMR (100 MHz, CD₃OD) δ 161.3, 148.7, 136.9, 133.7, 133.0, 132.5, 132.2, 131.1, 129.3, 106.4; HRMS found [M+H]⁺ 217.0539, C₁₀H₉N₄S requires 217.0548.

### Example 8

This example describes the synthesis of 4-(thiophen-2-yl)-1H-imidazo[4,5-c]pyridin-2-amine, (I where R^{b}═NH₂, R^{d}=2-thiophene, R^{a}═R^{c}═H), in a one-pot reaction. A mixture of 2-aminohistamine (0.11 mmol) and 2-thiofurfural (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure 4-(thiophen-2-yl)-1H-imidazo[4,5-c]pyridin-2-amine (20.7 mg; 73%) as a white solid. ¹H NMR (400 MHz, CD₃OD) δ 8.10 (d, J=6.4 Hz, 1H), 8.09 (dd, J=3.8, 1.1 Hz, 1H), 7.88 (dd, J=5.0, 1.1 Hz, 1H), 7.48 (d, J=6.4 Hz, 1H), 7.32 (dd, J=5.0, 3.8 Hz, 1H)

### Example 9

This example describes the synthesis of 4-(furan-3-yl)-1H-imidazo[4,5-c]pyridin-2-amine, (I where R^{b}═NH₂, R^{d}=3-furan, R^{a}═R^{c}═H), in a one-pot reaction. A mixture of 2-aminohistamine (0.11 mmol) and 3-furfural (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure 4-(furan-2-yl)-1H-imidazo[4,5-c]pyridin-2-amine (16.7 mg; 76%) as a white solid. ¹H NMR (400 MHz, CD₃OD) δ 8.74 (m, 1H), 8.10 (d, J=6.4 Hz, 1H), 7.81 (m, 1H), 7.49 (d, J=6.4 Hz, 1H), 7.23 (m, 1H); ¹³C NMR (100 MHz, CD₃OD) δ 161.4, 148.5, 147.5, 146.2, 137.8, 132.8, 131.2, 118.0, 108.7, 106.4; m/z 201 (100; M+H⁺); HRMS found [M+H]⁺ 201.0771, C₁₀H₉N₄O requires 201.0776.

### Example 10

This example describes the two step synthesis of 4-(thiophen-3-yl)-1H-imidazo[4,5-c]pyridin-2-amine, (I where R^{b}═NH₂, R^{d}=3-thiophene, R^{a}═R^{c}═H), in two steps. To a stirred solution of 2-Boc-2-AH (0.11 mmol) in ethanol (5 mL) was added triethylamine (0.16 mmol) and 2-thiofurfural (0.13 mmol). The reaction mixture was stirred at rt for 3 h, after which the solvent was removed under reduced pressure and the reaction mixture was further subjected to flash chromatography over silica gel using a gradient of 5:95-30:70 (MeOH:DCM saturated with ammonia) yielding N″-tert-butyl-4-(thiophen-2-yl)-4,5,6,7-tetrahydro-1H-imidazo[4,5-c]pyridine-2-ylcarbamate. (28 mg; 80%) as a white solid. ¹H NMR (400 MHz, CDCl₃) δ 7.27-7.24 (m, 1H), 7.02 (dd, J=4.9, 1.3 Hz, 1H), 6.82-6.80 (m, 1H), 5.80 (bs, 1H), 5.23 (s, 1H), 2.99-2.92 (m, 1H), 2.86-2.78 (m, 1H), 2.60-2.50 (m, 1H), 2.41-2.33 (m, 1H), 2.20 (bs, 1H), 1.24 (s, 9H); ¹³C NMR (100 MHz, CDCl₃) δ 150.5, 150, 144.6, 134.2, 127.7, 125.4, 122.4, 118.8, 85.1, 52.0, 38.5, 27.4, 25.8; ir (KBr) 3447, 3106, 1724, 1647, 1388, 1346, 1259, 1142, 1107, 839, 748 cm⁻¹; HRMS found [M+H]⁺ 321.1397, C₁₅H₂₁N₄O₂S requires 321.1985. To a stirred solution of this tetrahydro-intermediate (III) (0.15 mmol) in chloroform (10 mL) was added chloranil (0.30 mmol) and the reaction mixture refluxed for 20 h. Solvent was removed on a rotary evaporator and the residue obtained further purified by flash chromatography over silica gel with a gradient of 5:95-15:85 (MeOH:DCM saturated with ammonia) giving pure 4-(thiophen-3-yl)-1H-imidazo[4,5-c]pyridin-2-amine (26 mg; 80%) as a white solid. ¹H NMR (400 MHz, CD₃OD) δ 8.60 (dd, J=2.9, 1.3 Hz, 1H), 8.11 (d, J=6.4 Hz, 1H), 7.90 (dd, J=5.2, 1.3 Hz, 1H), 7.73 (dd, J=5.1, 2.9 Hz, 1H), 7.51 (d, J=6.4 Hz, 1H); ¹³C NMR (100 MHz, CD₃OD) δ 161.9, 150.3, 137.0, 133.2, 132.7, 131.9, 130.8, 128.9, 127.2, 107.0; HRMS found [M+H]⁺ 217.0545, C₁₀H₉N₄S requires 217.0548.

### Example 11

This example describes the synthesis of 4-(thiophen-3-yl)-1H-imidazo[4,5-c]pyridin-2-amine, (I where R^{b}═NH₂, R^{d}=3-thiophene, R^{a}═R^{c}═H), in a one-pot reaction. A mixture of 2-aminohistamine (0.11 mmol) and 3-thiofurfural (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure 4-(thiophen-3-yl)-1H-imidazo[4,5-c]pyridin-2-amine (20.4 mg; 86%) as a white solid. ¹H NMR (400 MHz, CD₃OD) δ 8.60 (dd, J=2.9, 1.3 Hz, 1H), 8.11 (d, J=6.4 Hz, 1H), 7.90 (dd, J=5.2, 1.3 Hz, 1H), 7.73 (dd, J=5.1, 2.9 Hz, 1H), 7.51 (d, J=6.4 Hz, 1H).

### Example 12

This example describes the two step synthesis of 4-(pyrido-2-yl)-1H-imidazo[4,5-c]pyridin-2-amine, (I where R^{b}═NH₂, R^{d}=2-pyridine, R^{a}═R^{c}═H), in two steps. To a stirred solution of 2-AH (0.11 mmol) in ethanol (5 mL) was added triethylamine (0.16 mmol) and 2-picolinaldehyde (0.13 mmol). The reaction mixture was stirred at rt for 6 h, after which the solvent was removed under reduced pressure and the reaction mixture was further subjected to flash chromatography over silica gel using a gradient of 5:95-30:70 (MeOH:DCM saturated with ammonia) yielding 4-(pyrid-2-yl)-4,5,6,7-tetrahydro-1H-imidazo[4,5-c]pyridine-2-ylcarbamate. (21 mg; 89%) as a white solid. ¹H NMR 400 MHz, CD₃OD) δ 8.51 (ddd, J=4.9, 1.7, 0.9 Hz, 1H), 7.79-7.73 (m, 1H), 7.36-7.26 (m, 2H), 4.92 (t, J=1.8 Hz, 1H), 3.14-3.07 (m, 1H), 3.01-2.93 (m, 1H), 2.65-2.50 (m, 2H); ¹³C NMR (100 MHz, CD₃OD) δ 162.1, 150.7, 150.1, 138.4, 126.5, 124.9, 124.3, 124.0, 59.0, 42.0, 24.0; ir (KBr) 3404, 1620, 1573, 1473, 1435, 1092, 752 cm⁻¹; HRMS found [M+H]⁺216.1246, C₁₁H₁₄N₅ requires 216.1249. To a stirred solution of tetrahydro-intermediate (III) (0.15 mmol) in ethanol was added 10% Pd/C (30 mg) and the reaction mixture refluxed for 18-24 h. The mixture was then filtered through a celite pad and washed with methanol (3×25 mL) and toluene (2×20 mL). The combined filtrates were concentrated under reduced pressure and subject to flash chromatography over silica gel using a gradient of 5:95-20:80 (MeOH:DCM saturated with ammonia) yielding pure 4-(pyrid-2-yl)-1H-imidazo[4,5-c]pyridin-2-amine (27.5 mg; 87%) as a white solid. ¹H NMR (400 MHz, CD₃OD) δ 8.89-8.80 (m, 2H), 8.28 (d, J=6.2 Hz, 1H), 8.07-8.02 (m, 1H), 7.57-7.52 (m, 2H); ¹³C NMR (100 MHz, CD₃OD) δ 161.6, 160.5, 150.8, 150.0, 148.9, 139.0, 135.9, 133.8, 126.5, 125.0, 107.7; HRMS found [M+H]⁺ 212.0932, C₁₁H₁₀N₅ requires 212.0936.

### Example 13

This example describes the synthesis of 4-(pyrid-2-yl)-1H-imidazo[4,5-c]pyridin-2-amine, (I where R^{b}═NH₂, R^{d}=2-pyridine, R^{a}═R^{c}═H), in a one-pot reaction. A mixture of 2-aminohistamine (0.11 mmol) and 2-picolinaldehyde (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure 4-(pyrid-2-yl)-1H-imidazo[4,5-c]pyridin-2-amine (27.5 mg; 87%) as a white solid. NMR (400 MHz, CD₃OD) δ 8.89-8.80 (m, 2H), 8.28 (d, J=6.2 Hz, 1H), 8.07-8.02 (m, 1H), 7.57-7.52 (m, 2H).

### Example 14

This example describes the two step synthesis of 4-(pyrido-3-yl)-1H-imidazo[4,5-c]pyridin-2-amine, (I where R^{b}═NH₂, R^{d}=3-pyridine, R^{a}═R^{c}═H), in two steps. To a stirred solution of 2-AH (0.11 mmol) in ethanol (5 mL) was added triethylamine (0.16 mmol) and 3-picolinaldehyde (0.13 mmol). The reaction mixture was stirred at rt for 6 h, after which the solvent was removed under reduced pressure and the reaction mixture was further subjected to flash chromatography over silica gel using a gradient of 5:95-30:70 (MeOH:DCM saturated with ammonia) yielding 4-(pyrid-3-yl)-4,5,6,7-tetrahydro-1H-imidazo[4,5-c]pyridine-2-ylcarbamate. (18.7 mg; 79%) as a white solid. ¹H NMR 400 MHz, CD₃OD) δ 8.49-8.46 (m, 1H), 8.43 (dd, J=4.9, 1.6 Hz, 1H), 7.75-7.71 (m, 1H), 7.38 (ddd, J=7.8, 4.9, 0.8 Hz, 1H), 4.89 (t, J=1.7 Hz, 1H), 3.12-3.04 (m, 1H), 3.01-2.93 (m, 1H), 2.68-2.59 (m, 1H), 2.57-2.48 (m, 1H); ¹³C NMR (100 MHz, CD₃OD) δ 150.8, 150.5, 149.1, 139.9, 138.4, 127.1, 125, 124.7, 55.9, 42.2, 23.8; ir (KBr) 3317, 1620, 1574, 1475, 1425, 1094, 1028, 801, 713 cm⁻¹; HRMS found [M+H]⁺216.1241, C₁₁H₁₄N₅ requires 216.1249. To a stirred solution of tetrahydro-intermediate (III) (0.15 mmol) in ethanol was added 10% Pd/C (30 mg) and the reaction mixture refluxed for 18-24 h. The mixture was then filtered through a celite pad and washed with methanol (3×25 mL) and toluene (2×20 mL). The combined filtrates were concentrated under reduced pressure and subject to flash chromatography over silica gel using a gradient of 5:95-20:80 (MeOH:DCM saturated with ammonia) yielding pure 4-(pyrid-3-yl)-1H-imidazo[4,5-c]pyridin-2-amine (25.3 mg; 80%) as a white solid. ¹H NMR (400 MHz, CD₃OD) δ 9.28 (d, J=1.9 Hz, 1H), 8.79 (dd, J=5.0, 1.5 Hz, 1H), 8.59-8.55 (m, 1H), 8.30 (d, J=6.6 Hz, 1H), 7.76 (ddd, J=8.0, 5.0, 0.8 Hz, 1H), 7.60 (d, J=6.6 Hz, 1H); ¹³C NMR (100 MHz, CD₃OD) δ 162.0, 151.5, 150.0, 149.7, 138.8, 137.6, 135.4, 133.8, 129.1, 125.7, 107.9; HRMS found [M+H]⁺ 212.0928, C₁₁H₁₀N₅ requires 212.0936.

### Example 15

This example describes the synthesis of 4-(pyrid-3-yl)-1H-imidazo[4,5-c]pyridin-2-amine, (I where R^{b}═NH₂, R^{d}=3-pyridine, R^{a}═R^{c}═H), in a one-pot reaction. A mixture of 2-aminohistamine (0.11 mmol) and 3-picolinaldehyde (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure 4-(pyrid-3-yl)-1H-imidazo[4,5-c]pyridin-2-amine (17.4 mg; 75%) as a white solid. ¹H NMR (400 MHz, CD₃OD) δ 9.28 (d, J=1.9 Hz, 1H), 8.79 (dd, J=5.0, 1.5 Hz, 1H), 8.59-8.55 (m, 1H), 8.30 (d, J=6.6 Hz, 1H), 7.76 (ddd, J=8.0, 5.0, 0.8 Hz, 1H), 7.60 (d, J=6.6 Hz, 1H).

### Example 16

This example describes the two step synthesis of 4-(quinolin-3-yl)-1H-imidazo[4,5-c]pyridin-2-amine, (I where R^{b}═NH₂, R^{d}=3-quinoline, R^{a}═R^{c}═H), in two steps. To a stirred solution of 2-AH (0.11 mmol) in ethanol (5 mL) was added triethylamine (0.16 mmol) and 3-quinoline carboxaldehyde (0.13 mmol). The reaction mixture was stirred at rt for 6 h, after which the solvent was removed under reduced pressure and the reaction mixture was further subjected to flash chromatography over silica gel using a gradient of 5:95-30:70 (MeOH:DCM saturated with ammonia) yielding 4-(quinolin-3-yl)-4,5,6,7-tetrahydro-1H-imidazo[4,5-c]pyridine-2-ylcarbamate. (18.7 mg; 79%) as a white solid. ¹H NMR 400 MHz, CD₃OD) δ 8.82 (d, J=2.1 Hz, 1H), 8.17 (d, J=2.1 Hz, 1H), 8.01 (dd, J=8.5, 0.9 Hz, 1H), 7.89 (dd, J=8.1, 1.4 Hz, 1H), 7.75-7.70 (m, 1H), 7.60-7.55 (m, 1H), 5.00 (t, J=1.8 Hz, 1H), 3.16-3.08 (m, 1H), 3.05-2.98 (m, 1H), 2.73-2.64 (m, 1H), 2.61-2.52 (m, 1H); ¹³C NMR (100 MHz, CD₃OD) δ 152.4, 150.7, 148.2, 137.5, 136.5, 130.9, 129.4, 129.3, 128.9, 128.2, 126.8, 124.6, 55.9, 42.2, 23.8; ir (KBr) 3422, 2925, 1620, 1573, 1473, 1124, 861, 752 cm⁻¹; HRMS found [M+H]⁺ 266.1406, C₁₅H₁₆N₅ requires 266.1406. To a stirred solution of tetrahydro-intermediate (III) (0.15 mmol) in ethanol was added 10% Pd/C (30 mg) and the reaction mixture refluxed for 18-24 h. The mixture was then filtered through a celite pad and washed with methanol (3×25 mL) and toluene (2×20 mL). The combined filtrates were concentrated under reduced pressure and subject to flash chromatography over silica gel using a gradient of 5:95-20:80 (MeOH:DCM saturated with ammonia) yielding pure 4-(quinolin-3-yl)-1H-imidazo[4,5-c]pyridin-2-amine (25.3 mg; 74%) as a white solid. ¹H NMR (400 MHz, CD₃OD) δ 9.53 (d, J=2.2 Hz, 1H), 9.11 (d, J=2.2 Hz, 1H), 8.33 (d, J=6.4 Hz, 1H), 8.18-8.12 (m, 2H), 7.98-7.92 (m, 1H), 7.80-7.75 (m, 1H), 7.62 (d, J=6.3 Hz, 1H); ¹³C NMR (100 MHz, CD₃OD) δ 161.8, 149.7, 148.3, 139.7, 137.6, 135.8, 135.5, 133.5, 130.2, 129.5, 128.8, 128.7, 125.5, 107.9; HRMS found [M+H]⁺ 262.1091, C₁₅H₁₂N₅ requires 262.1093.

### Example 17

This example describes the synthesis of 4-(quinolin-3-yl)-1H-imidazo[4,5-c]pyridin-2-amine, (I where R^{b}═NH₂, R^{d}=3-quinoline, R^{a}═R^{c}═H), in a one-pot reaction. A mixture of 2-aminohistamine (0.11 mmol) and 3-quinoline carboxaldehyde (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure 4-(quinolin-3-yl)-1H-imidazo[4,5-c]pyridin-2-amine (20 mg; 70%) as a white solid. ¹H NMR (400 MHz, CD₃OD) δ 9.53 (d, J=2.2 Hz, 1H), 9.11 (d, J=2.2 Hz, 1H), 8.33 (d, J=6.4 Hz, 1H), 8.18-8.12 (m, 2H), 7.98-7.92 (m, 1H), 7.80-7.75 (m, 1H), 7.62 (d, J=6.3 Hz, 1H).

### Example 18

This example describes the two step synthesis of 4-(benzothiophen-3-yl)-1H-imidazo[4,5-c]pyridin-2-amine, (I where R^{b}═NH₂, R^{d}=3-benzothiophene, R^{a}═R^{c}═H), in two steps. To a stirred solution of N-Boc-2-AH (0.11 mmol) in ethanol (5 mL) was added triethylamine (0.16 mmol) and 3-benzothiophene carboxaldehyde (0.13 mmol). The reaction mixture was stirred at rt for 6 h, after which the solvent was removed under reduced pressure and the reaction mixture was further subjected to flash chromatography over silica gel using a gradient of 5:95-30:70 (MeOH:DCM saturated with ammonia) yielding N″-tert-butyl-4-(benzothiophen-3-yl)-4,5,6,7-tetrahydro-1H-imidazo[4,5-c]pyridine-2-ylcarbamate. (28.5 mg; 70%) as a white solid. ¹HNMR (400 MHz, CDCl₃) δ 7.94 (d, J=7.7 Hz, 1H), 7.85-7.81 (m, 1H), 7.42-7.29 (m, 2H), 6.83 (s, 1H), 5.86 (bs, 1H), 5.57 (s, 1H), 2.96 (ddd, J=13.1, 5.9, 2.1 Hz, 1H), 2.82 (ddd, J=13.1, 10.3, 4.7 Hz, 1H), 2.63-2.53 (m, 1H), 2.43-2.35 (m, 1H), 2.17 (bs, 1H), 1.02 (s, 9H); ¹³C NMR (100 MHz, CDCl₃) δ 150.8, 150.0, 140.8, 137.9, 134.9, 124.4, 124.2, 124.1, 123.0, 122.2, 117.8, 85.1, 50.8, 38.6, 27.4, 25.8; ir (KBr) 3453, 2928, 1722, 1627, 1369, 1326, 1263, 1138, 835, 765, 734 cm⁻¹; HRMS found [M+H]⁺ 371.1547, C₁₉H₂₃N₄O₂S requires 371.1542. To a stirred solution of this tetrahydro-intermediate (III) (0.15 mmol) in chloroform (10 mL) was added chloranil (0.30 mmol) and the reaction mixture refluxed for 20 h. Solvent was removed on a rotary evaporator and the residue obtained further purified by flash chromatography over silica gel with a gradient of 5:95-15:85 (MeOH:DCM saturated with ammonia) giving pure 4-(benzothiophen-3-yl)-1H-imidazo[4,5-c]pyridin-2-amine (25.5 mg; 64%) as a white solid. ¹H NMR (400 MHz, CD₃OD) δ 8.29 (s, 1H), 8.27 (d, J=6.5 Hz, 1H), 8.10-8.07 (m, 1H), 7.79-7.75 (m, 1H), 7.60 (d, J=6.5 Hz, 1H), 7.55-7.48 (m, 2H); ¹³C NMR (100 MHz, CD₃OD) δ 159.2, 158.8, 151.7, 141.7, 137.8, 135.8, 133.3, 126.9, 126.6, 124.2, 123.5, 117.4, 114.6, 109.0; HRMS found [M+H]⁺ 267.0706, C₁₄H₁₁N₄S requires 267.0704.

### Example 19

This example describes the synthesis of 4-(benzothiophen-3-yl)-1H-imidazo[4,5-c]pyridin-2-amine, (I where R^{b}═NH₂, R^{d}=3-benzothiophene, R^{a}═R^{c}═H), in a one-pot reaction. A mixture of 2-aminohistamine (0.11 mmol) and 3-benzothiophene carboxaldehyde (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure 4-(benzothiophen-3-yl)-1H-imidazo[4,5-c]pyridin-2-amine (22.2 mg; 76%) as a white solid. ¹H NMR (400 MHz, CD₃OD) δ 8.29 (s, 1H), 8.27 (d, J=6.5 Hz, 1H), 8.10-8.07 (m, 1H), 7.79-7.75 (m, 1H), 7.60 (d, J=6.5 Hz, 1H), 7.55-7.48 (m, 2H).

### Example 20

This example describes the synthesis of 4-phenyl-1H-imidazo[4,5-c]pyridin-2-amine, (I where R^{b}═NH₂, R^{d}=phenyl, R^{a}═R^{c}═H), in a one-pot reaction. A mixture of 2-aminohistamine (0.11 mmol) and benzaldehyde (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure 4-phenyl-1H-imidazo[4,5-c]pyridin-2-amine (13.6 mg; 59%) as a white solid. ¹H NMR (400 MHz, CD₃OD) δ 8.13 (d, J=5.4 Hz, 1H), 7.90 (d, J=7.4 Hz, 2H), 7.54-7.48 (m, 2H), 7.46-7.41 (m, 1H), 7.19 (d, J=5.5 Hz, 1H); ¹³C NMR (100 MHz, CD₃OD) δ 148.8, 136.3, 134.9, 131.9, 129.7, 129.4, 128.9, 120.0, 117.1, 114.2, 111.4, 107.5; m/z 211 (100; M+H⁺); HRMS found (M+H)⁺ 211.0987, C₁₂H₁₁N₄ requires 211.0984.

### Example 21

This example describes the synthesis of 4-(pyrid-2-yl)-1H-imidazo[4,5-c]pyridine, (I where R^{b}═H, R^{d}=2-pyridyl, R^{a}═R^{c}═H), in two steps. A mixture of histamine (0.11 mmol), potassium hydroxide (0.33 mmol) and 2-formylpyridine (0.13 mmol) were refluxed in ethanol for 48 h, after which the solvent was evaporated and the product chromatographed on silica (1% methanol/DCM to 12% methanol saturated with ammonia) to yield 4-(pyrid-2-yl)-4,5,6,7-tetrahydro-1H-imidazo[4,5-c]pyridine (18.8 mg; 20%) as a white solid. ¹H NMR (400 MHz, CD₃OD) δ 8.49 (d, J=5.4 Hz, 1H), 7.87 (t, J=5.4 Hz, 1H), 7.62 (s, 1H), 7.54-7.40 (m, 2H),3.3-2.7 (m, 4H); ¹³C NMR (100 MHz, CD₃OD) δ 160.0, 149.4, 138.8, 135.7, 131.4 (b), 128.3 (b), 123.0, 58.2, 49.5, 41.0, 22.6; m/z 201 (100; M+H⁺).

4-(Pyrid-2-yl)-4,5,6,7-tetrahydro-1H-imidazo[4,5-c]pyridine (0.05 mmol) and IBX (0.075 mmol) were dissolved in DMSO (300 μL) and heated to 45° C. for 7 hours. Flash chromatography on silica gel (1% methanol/DCM to 12% methanol saturated with ammonia) yielded 4-(pyrid-2-yl)-1H-imidazo[4,5-c]pyridine (9 mg; 90%) as a pale yellow solid ¹H NMR (400 MHz, CD₃OD) δ 8.74 (m, 2H), 8.55 (d, J=5.4 Hz, 1H), 8.32 (s, 1H), 7.92 (t, 1H), 7.85 (d, 1H), 7.38 (dd, 1H); m/z 197 (100; M+H⁺)

### Example 22

This example describes the synthesis of 6-carboxy-4-(furyl-2-yl)-1H-imidazo[4,5-c]pyridine, (I where R^{b}═H, R^{d}=2-furyl, R^{a}═—COOH, R^{c}═H), in two steps. A mixture of histidine (0.5 mmol), potassium hydroxide (1.5 mmol) and 2-furfural (0.6 mmol) were refluxed in aqueous ethanol for 48 h, after which the solvent was evaporated and the product chromatographed on silica (1% methanol/DCM to 12% methanol saturated with ammonia) to yield 6-carboxy-4-(furyl-2-yl)-4,5,6,7-tetrahydro-1H-imidazo[4,5-c]pyridine (66 mg; 56%) as a mixture of diasteromers. NMR (400 MHz, CD₃OD) 7.70/7.67 (s, 1H), 7.49/7.50 (s, 1H), 6.58/6.46 (d, 1H), 6.48/6.28 (d, 1H), 5.60/5.55 (bs, 1H), 4.0-3.9 (m, 1H), 3.24-3.16 (M, 1H), 2.98-2.87 (m, 1H); ¹³C NMR (100 MHz, CD₃OD) δ 175.5, 174.7, 149.7, 148.2, 144.9, 144.8, 136.8, 127.8, 127.3, 125.5, 125.5, 112.2, 112.1, 111.3, 111.2, 57.9, 53.7, 51.7, 49.5, 49.3, 24.1, 24.0.

Alternatively, a mixture of histidine (75 mg, 0.5 mmol), potassium carbonate (775 mg, 5.6 mmol) and 2-furfural (45 mg, 0.6 mmol) were refluxed in absolute ethanol containing finely powdered, freshly activated molecular sieves (750 mg) and the reaction mixture refluxed for 1 day after which it was filtered and the residue washed with methanol (20 mL). The filtrate was evaporated in vacuo and the yellow residue subjected to column chromatography over silica using a gradient of 10:90-40:60 (MeOH:CHCl₃ saturated with ammonia) to yield a diastereomeric mixture of the tetrahydro-intermediate (III) as a brown crystals (105 mg, 90%). ¹H NMR (400 MHz, D₂O) δ major diastereomer: 7.71 (s, 1H), 7.50 (dd, J=1.8, 0.7 Hz, 1H), 6.40 (dd, J=3.3, 1.8, 1H), 6.28 (d, J=3.4 Hz, 1H), 5.60 (s, 1H), 3.95-3.91 (m, 1H), 3.24-3.16 (m, 1H), 2.98-2.87 (m, 1H); minor diastereomer: 6.67 (s, 1H), 7.51 (dd, J=1.8, 0.9 Hz, 1H), 6.58 (d, J=3.1 Hz, 1H), 6.47 (dd, J=3.4, 1.9 Hz, 1H), 5.60 (s, 1H), 4.01-3.97 (m, 1H), 3.24-3.16 (m, 1H), 2.98-2.87 (m, 1H); ¹³C NMR (100 MHz, D₂O) δ major diastereomer: 175.5, 149.7, 144.83, 136.9, 127.3, 125.4, 112.17, 111.26, 53.7, 51.6, 24.0; minor diastereomer: 174.7, 148.2, 144.88, 136.8, 127.8, 125.7, 112.21, 111.31, 57.9, 49.3, 24.1; HRMS found [M+H]⁺ 234.0874, C₁₁H₁₂N₃O₃ requires 234.0873.

6-carboxy-4-(furyl-2-yl)-4,5,6,7-tetrahydro-1H-imidazo[4,5-c]pyridine (III) (0.1 mmol) and IBX (0.16 mmol) were dissolved in DMSO (500 μL) and heated to 45° C. for 6 hours. Flash chromatography on silica gel (1% methanol/DCM to 12% methanol saturated with ammonia) yielded 6-carboxy-4-(furyl-2-yl)-1H-imidazo[4,5-c]pyridine (4.6 mg; 20%) as a white solid ¹H NMR (400 MHz, CD₃OD) δ 8.35 (s, 1H), 8.02 (s, 1H), 7.79 (bd, 1H), 7.40 (d, 1H), 6.72 (dd, 1H); m/z 230 (100; M+H⁺)

Alternatively, 6-carboxy-4-(furyl-2-yl)-4,5,6,7-tetrahydro-1H-imidazo[4,5-c]pyridine was heated with sulfur (S₈/DMF, 140° C., 2 h) to yield 4-(furyl-2-yl)-1H-imidazo[4,5-c]pyridine, (I where R^{b}═H, R^{d}=2-furyl, R^{a}═R^{c}═H), where dehydrogenation was effected with concomitant decarboxylation to yield the ageladine analog (I where R^{d}=2-furan, R^{a}═R^{b}═H), which was isolated in 28% yield. ¹H NMR (400 MHz, DMSO-d₆) δ 8.86 (s, 1H), 8.44 (d, J=6.4 Hz, 1H), 8.26 (dd, J=1.7, 0.7 Hz, 1H), 8.03 (d, J=3.48 Hz, 1H), 7.94 (d, J=6.2 Hz, 1H), 6.97 (dd, J=1.8, 3.4 Hz, 1H); ¹³C NMR (100 MHz, DMSO-d₆) δ 149.1, 148.2, 145.9, 144.0, 135.9, 135.1, 134.7, 119.2, 114.1, 109.7; HRMS found [M+H]⁺ 186.0664, C₁₀H₈N₃O requires 186.0662.

### Example 23

This example describes the two step synthesis of indole analog, (I where R^{b}═NH₂, R^{d}=3-indole, R^{a}═R^{c}═H), in two steps. To a stirred solution of 2-AH (0.11 mmol) in ethanol (5 mL) was added triethylamine (0.16 mmol) and N-Boc-3-indole carboxaldehyde (0.13 mmol). The reaction mixture was stirred at rt for 6 h, after which the solvent was removed under reduced pressure and the reaction mixture was further subjected to flash chromatography over silica gel using a gradient of 5:95-30:70 (MeOH:DCM saturated with ammonia) yielding N″-tert-Butyl-3-(2-amino-4,5,6,7-tetrahydro-1H-imidazo[4,5-c]pyridine-4-yl)1H-indole-1-carboxylate. (21.7 mg; 56%) as a white solid. ¹H NMR (400 MHz, CDCl₃) δ 8.03 (d, J=8.3 Hz, 1H), 7.42 (d, J=7.7 Hz, 1H), 7.36 (s, 1H), 7.24-7.19 (m, 1H), 7.13-7.08 (m, 1H), 4.99 (s, 1H), 3.11-3.00 (m, 1H), 2.93-2.82 (m, 1H), 2.50-2.27 (m, 2H), 1.59 (s, 9H); ¹³C NMR (100 MHz, CDCl₃) δ 149.7, 148.1, 135.8, 129.4, 124.5, 124.4, 122.6, 121.9, 119.7, 115.2, 83.7, 49.9, 49.0, 41.6, 28.2, 23.7; ir (KBr) 3334, 2977, 1735, 1635, 1562, 1453, 1371, 1308, 1255, 1154, 1091, 855, 747 cm⁻¹; HRMS found [M+H]⁺ 354.1940, C₁₉H₂₄N₅O₂ requires 354.1930. To a stirred solution of this tetrahydro-intermediate (III) (0.15 mmol) in chloroform (10 mL) was added chloranil (0.30 mmol) and the reaction mixture refluxed for 20 h. Solvent was removed on a rotary evaporator and the residue obtained further purified by flash chromatography over silica gel with a gradient of 5:95-15:85 (MeOH:DCM saturated with ammonia) giving pure 4-(1H-indole-3-yl)-1H-imidazo[4,5-c]pyridine-2-amine (26.5 mg; 71%) as a white solid. ¹H NMR (400 MHz, CD₃OD) δ 8.15 (s, 1H), 8.12 (d, J=6.59 Hz, 1H), 7.87-7.83 (m, 1H), 7.79-7.55 (m, 1H), 7.46 (d, J=6.6 Hz, 1H), 7.46 (d, J=6.6 Hz, 1H), 7.34-7.23 (m, 2H); ¹³C NMR (100 MHz, CD₃OD) δ 162.0, 138.4, 134.3, 133.5, 130.2, 125.7, 124.4, 122.5, 120.1, 113.4, 107.3, 106.1; HRMS found [M+H]⁺ 250.1087, C₁₄H₁₂N₅ requires 250.1093.

### Example 24

This example describes the synthesis of indole analog of ageladine A, (I where R^{b}═NH₂, R^{d}=3-indole, R^{a}═R^{c}═H), in a one-pot reaction. A mixture of 2-aminohistamine (0.11 mmol) and N-Boc-3-indole carboxaldehyde (0.13 mmol) was stirred in ethanol at room temperature for 6 h, after which 10% Pd/C (24 mg) was added and the reaction mixture refluxed for a further 24 h. The mixture was then filtered through a celite pad, washed with ethanol (3×25 mL) and toluene (2×20 mL). The combined filtrates were concentrated under reduced pressure and chromatographed over silica gel using a gradient of 5:95-20:80 giving pure didebromoageladine A (9.8 mg; 36%) as a white solid. ¹H NMR (400 MHz, CD₃OD) δ 7.93 (d, J=6.5 Hz, 1H), 7.34 (d, J=6.5 Hz, 1H), 7.23 (dd, J=2.6, 1.4 Hz, 1H), 7.18 (dd, J=4.0, 1.4 Hz, 1H), 6.42 (dd, J=3.9, 2.6 Hz, 1H).

### Example 25

This example describes the two step synthesis of the 2-pyridine analog, (I where R^{b}═H, R^{d}=2-pyridine, R^{a}═COOH, R^{c}═H), in two steps. To a stirred solution of histidine (72.4 mg, 0.467 mmol.) in ethanol (10 mL) were added pyridine-2-carboxaldehyde (50.0 mg, 0.467 mmol.) followed by potassium carbonate (774.2 mg, 5.60 mmol.) and 3 Å molecular sieves (724 mg). The reaction mixture was refluxed (80° C.) for 1 day after which it was filtered and the residue washed with methanol (20 mL). The filtrate was evaporated in vacuo and the yellow residue subjected to column chromatography over silica using a gradient of 10:90-40:60 (MeOH:CHCl₃ saturated with ammonia) to yield a diastereomeric mixture of the tetrahydro-intermediate (III) as a clear solid in quantitative yield. ¹H NMR (400 MHz, DMSO-d₆) δ major diastereomer: 8.56-8.55 (m, 1H), 7.83-7.77 (m, 1H), 7.55-7.53 (m, 1H), 7.56 (s, 1H), 7.36-7.32 (m, 1H), 5.32 (s, 1H), 3.69-3.64 (m, 1H), 2.98-2.91 (m, 1H), 2.83-2.70 (m, 1H) minor diastereomer: 8.60-8.58 (m, 1H), 7.83-7.77 (m, 1H), 7.56 (s, 1H), 7.45-7.42 (m, 1H), 7.36-7.32 (m, 1H), 5.22 (s, 1H), 3.69-3.64 (m, 1H), 2.98-2.91 (m, 1H); ¹³C NMR (100 MHz, DMSO-d₆) δ major diastereomer: 173.9, 160.5, 149.4, 137.5, 135.2, 132.6, 126.8, 123.9, 123.7, 57.7, 54.1, 27.1; minor diastereomer: 173.2, 160.3, 149.7, 137.6, 135.7, 130.9, 126.8, 123.55, 123.51, 59.3, 56.5, 25.9; HRMS found [M+H]⁺ 245.1033, C₁₂H₁₃N₄O₂ requires 245.1033.

The tetrahydro-intermediate (III) was dehydrogenated with IBX (1.5 equiv.) in DMSO at 45° C. for 6 hrs. DMSO was removed on the freeze-drier to yield a yellow crude residue which was first purified using column chromatography over silica using a gradient of 5:95-45:55 (MeOH:CHCl₃ saturated with ammonia) then by HPLC (gradient 0-70% acetonitrile/water, containing 0.1% TFA) to yield the TFA salt of 4-(pyridin-2-yl)-3H-imidazo[4,5-c]pyridine-6-carboxylic acid, (I) (where R^{b}═H, R^{d}=2-pyridine, R^{a}═COOH, R^{c}═H) (68%) as a white solid. ¹H NMR (400 MHz, DMSO-d₆) δ 8.91-8.89 (m, 2H), 8.76 (s, 1H), 8.48 (s, 1H), 8.20 (dt, J=7.8, 1.6 Hz, 1H), 7.67 (dd, J=7.0, 5.3 Hz, 1H); ¹³C NMR (100 MHz, DMSO-d₆) δ 167.1, 154.8, 149.4, 149.3, 148.7, 140.8, 140.7, 133.6, 139.5, 125.7, 123.1, 116.7; HRMS found [M+H]⁺ 241.0714, C₁₂H₉N₄O₂ requires 241.0725. The decarboxylated analog, 4-(pyridin-2-yl)-3H-imidazo[4,5-c]pyridine, (I) (where R^{d}=2-pyridine, R^{a}═R^{b}═R^{c}═H) was isolated in 28% yield. ¹H NMR (400 MHz, CD₃OD) δ 9.52 (d, J=7.2 Hz, 1H), 8.98 (bs, 1H), 8.91 (s, 1H), 8.61 (d, J=5.9 Hz, 1H), 8.23 (t, J=7.7 Hz, 1H), 8.19 (d, J=5.9 Hz, 1H), 7.75 (bs, 1H); ¹³C NMR (100 MHz, CDCl₃) δ 150.1, 148.9, 147.4, 146.5, 140.0, 138.6, 135.9, 134.7, 127.0, 126.5, 111.99; HRMS found [M+H]⁺ 197.0823, C₁₁H₉N₄ requires 197.0827.

### Example 26

This example describes the two step synthesis of the 3-pyridine analog, (I where R^{b}═H, R^{d}=3-pyridine, R^{a}═COOH, R^{c}═H), in two steps. To a stirred solution of histidine (72.4 mg, 0.467 mmol.) in ethanol (10 mL) were added pyridine-2-carboxaldehyde (50.0 mg, 0.467 mmol.) followed by potassium carbonate (774.2 mg, 5.60 mmol.) and 3 Å molecular sieves (724 mg). The reaction mixture was refluxed (80° C.) for 1 day after which it was filtered and the residue washed with methanol (20 mL). The filtrate was evaporated in vacuo and the yellow residue subjected to column chromatography over silica using a gradient of 10:90-40:60 (MeOH:CHCl₃ saturated with ammonia) to yield a diastereomeric mixture of the tetrahydro-intermediate (III) as a clear solid (99%). ¹H NMR (400 MHz, DMSO-d₆) δ major diastereomer: 8.60 (dd, J=4.9, 1.6 Hz, 1H), 8.58-8.57 (m, 1H), 7.93-7.75 (m, 2H), 7.54-7.48 (m, 1H), 5.51 (s, 1H), 4.09-4.05 (m, 1H), 3.31-3.25 (m, 1H), 3.08-2.99 (m, 1H); minor diastereomer: 8.55 (dd, J=4.9, 1.6 Hz, 1H), 8.50-8.49 (m, 1H), 7.93-7.75 (m, 2H), 7.54-7.48 (m, 1H), 5.66 (s, 1H), 3.91-3.88 (m, 1H), 3.31-3.25 (m, 1H), 3.08-2.99 (m, 1H); ¹³C NMR (100 MHz, DMSO-d₆) δ major diastereomer: 175.7, 150.3, 150.0, 138.9, 138.8, 133.5, 129.9, 126.6, 125.2, 58.7, 53.6, 24.8; minor diastereomer: 176.3, 150.0, 149.8, 137.1, 136.9, 134.4, 128.6, 126.3, 125.1, 56.5, 53.6, 24.4; HRMS found [M+H]⁺ 245.1035, C₁₂H₁₃N₄O₂ requires 245.1033.

The tetrahydro-intermediate (III) (mixture of diasteromers) was dehydrogenated with IBX (1.5 equiv.) in DMSO at 45° C. for 6 hrs. DMSO was removed on the freeze-drier to yield a yellow crude residue which was first purified using column chromatography over silica using a gradient of 5:95-45:55 (MeOH:CHCl₃ saturated with ammonia) then by HPLC (gradient 0-70% acetonitrile/water/0.1% TFA) to yield the TFA salt as a white solid (60%). ¹H NMR (400 MHz, D₂O) δ 8.97 (bs, 1H), 8.47 (bs, 1H), 8.32 (bd, 1H), 8.14 (bs, 1H), 7.92 (bs, 1H), 7.46 (bt, 1H); ¹³C NMR (100 MHz, D₂O) δ 172.9, 149.0, 148.8, 146.3, 144.0, 141.4, 138.7, 138.4, 136.7, 133.1, 124.7, 109.8; HRMS found [M+H]⁺ 241.0722, C₁₂H₉N₄O₂ requires 241.0725. The analog without the carboxylic group (I) (where ═R^{d}=3-pyridine, R^{a}═R^{b}═R^{c}═H) was also isolated (35%) as a minor product. ¹H NMR (400 MHz, MeOD-d₄) δ 9.82 (bs, 1H), 9.18 (d, J=8.1 Hz, 1H), 8.99 (bs, 1H), 8.74 (bs, 1H), 8.635 (d, J=6.2 Hz, 1H), 8.06 (d, J=6.5 Hz, 1H), 8.02 (bs, 1H); ¹³C NMR (100 MHz, MeOD-d₄) δ 162.4(2C), 150.0, 149.9, 149.0, 145.4, 143.9, 142.9, 140.3, 139.0, 133.0, 127.8, 111.8; HRMS found [M+H]⁺ 197.0823, C₁₁H₉N₄ requires 197.0827.

### Example 27

This example describes the two step synthesis of the 2-imidazole analog, (I where R^{b}═H, R^{d}=2-imidazole, R^{a}═COOH, R^{c}═H), in two steps. To a stirred solution of histidine (72.4 mg, 0.467 mmol.) in ethanol (10 mL) were added imidazole-2-carboxaldehyde (37.4 mg, 0.467 mmol.) followed by potassium carbonate (774.2 mg, 5.60 mmol) and 3 Å molecular sieves (724 mg). The reaction mixture was refluxed (80° C.) for 1 day after which it was filtered and the residue washed with methanol (20 mL). The filtrate was evaporated in vacuo and the yellow residue subjected to column chromatography over silica using a gradient of 10:90-40:60 (MeOH:CHCl₃ saturated with ammonia) to yield a diastereomeric mixture of the tetrahydro-intermediate (III) as a white solid (85%). ¹H NMR (400 MHz, DMSO-d₆) δ major diastereomer: 7.50 (s, 1H), 6.95 (s, 2H), 5.28 (s, 1H), 3.86-3.82 (m, 1H), 2.95-2.85 (m, 2H); minor diastereomer: 7.46 (s, 1H), 6.94 (s, 2H), 5.19 (s, 1H), 3.62-3.58 (m, 1H), 2.77-2.71 (m, 2H); ¹³C NMR (100 MHz, DMSO-d₆) δ major diastereomer: 174.4, 148.3, 136.0(2C), 135.3, 122.4, 54.1, 50.2, 26.5; minor diastereomer: 173.9, 147.9, 135.9(2C), 135.0, 122.6, 57.9, 52.7, 27.3; HRMS found [M+H]⁺ 234.0986, C₁₀H₁₂N₅O₂ requires 234.0986.

The tetrahydro-intermediate (III) (mixture of diasteromers) was dehydrogenated with IBX (1.5 equiv.) in DMSO at 45° C. for 6 hrs. DMSO was removed on the freeze-drier to yield a yellow crude residue which was first purified using column chromatography over silica using a gradient of 5:95-45:55 (MeOH:CHCl₃ saturated with ammonia) then by HPLC (gradient 0-70% acetonitrile/water/0.1% TFA) to yield the TFA salt of the analog (58%) as a white solid. ¹H NMR (400 MHz, DMSO-d₆) δ 8.88 (s, 1H), 8.47 (s, 1H), 7.81 (s, 2H); ¹³C NMR (100 MHz, DMSO-d₆) δ 166.2, 149.6, 144.1, 142.0, 140.4, 137.1, 132.6, 123.4(2C), 113.3; HRMS found [M+H]⁺ 230.0674, C₁₀H₈N₅O₂ requires 230.0673. The reaction also yielded the decarboxylated product, 4-(1H-imidazol-2-yl)-3H-imidazo[4,5-c]pyridine, (I) (where ═R^{d}=2-imidazole, R^{a}═R^{b}═R^{c}═H) as a minor product (38%). ¹H NMR (400 MHz, DMSO-d₆) δ 8.83 (s, 1H), 8.575 (d, J=5.5 Hz, 1H), 7.925 (d, J=5.5 Hz, 1H), 7.79 (s, 2H); ¹³C NMR (100 MHz, DMSO-d₆) δ 147.7, 142.0, 141.8, 141.7, 137.2, 133.3, 122.8(2C), 111.7; HRMS found [M+H]⁺ 186.0776, C₉H₈N₅ requires 186.0774.

### Example 28

This example describes the two step synthesis of the 2-thiophene analog, (I where R^{b}═H, R^{d}=2-thiophene, R^{a}═COOH, R^{c}═H), in two steps. To a stirred solution of histidine (72.4 mg, 0.467 mmol.) in ethanol (10 mL) were added thiophene-3-carboxaldehyde (52.3 mg, 0.467 mmol.) followed by potassium carbonate (774.2 mg, 5.60 mmol.) and 3 Å molecular sieves (724 mg). The reaction mixture was refluxed (80° C.) for 1 day after which it was filtered and the residue washed with methanol (20 mL). The filtrate was evaporated in vacuo and the yellow residue subjected to column chromatography over silica using a gradient of 10:90-40:60 (MeOH:CHCl₃ saturated with ammonia) to yield a diastereomeric mixture of the tetrahydro-intermediate (III) as a white solid (86%). ¹H NMR (400 MHz, D₂O) δ major diastereomer: 7.74 (s, 1H), 7.13-7.08 (m, 2H), 5.84 (s, 1H), 4.03-3.99 (m, 1H), 3.26-3.20 (m, 1H), 3.02-2.92 (m, 1H); minor diastereomer: 7.70 (s, 1H), 7.28-7.27 (m, 1H), 7.12-7.07 (m, 1H), 5.74 (s, 1H), 4.03-3.99 (m, 1H), 3.26-3.20 (m, 1H), 3.02-2.92 (m, 1H); ¹³C NMR (100 MHz, D₂O) δ major diastereomer: 175.5, 139.3, 136.9, 130.3, 129.8, 128.2, 127.9, 126.4, 53.7, 51.2, 24.8; minor diastereomer: 176.1, 141.3, 136.9, 130.1, 129.2, 128.1, 128.05, 125.6, 58.6, 53.5, 24.5; HRMS found [M+H]⁺ found [M+H] 250.0646, C₁₁H₁₂N₃O₂S requires 250.0645.

### Example 29

This example describes the two step synthesis of the 3-thiophene analog, (I where R^{b}═H, R^{d}=3-thiophene, R^{a}═COOH, R^{c}═H), in two steps. To a stirred solution of histidine (72.4 mg, 0.467 mmol.) in ethanol (10 mL) were added thiophene-3-carboxaldehyde (52.3 mg, 0.467 mmol.) followed by potassium carbonate (774.2 mg, 5.60 mmol.) and 3 Å molecular sieves (724 mg). The reaction mixture was refluxed (80° C.) for 1 day after which it was filtered and the residue washed with methanol (20 mL). The filtrate was evaporated in vacuo and the yellow residue subjected to column chromatography over silica using a gradient of 10:90-40:60 (MeOH:CHCl₃ saturated with ammonia) to yield a diastereomeric mixture of the tetrahydro-intermediate (III) as a white solid (99%). ¹H NMR (400 MHz, D₂O) δ major diastereomer: 7.66 (s, 1H), 7.56 (m, 1H), 7.31-7.30 (m, 1H),7.07-7.04 (m, 1H), 5.68 (s, 1H), 4.05-4.00 (m, 1H), 3.25 (m, 1H), (2.99 (m, 1H); minor diastereomer: 7.71 (s, 1H), 7.55-7.45 (m, 1H), 7.31-7.30 (m, 1H), 7.07-7.04 (m, 1H), 5.56 (s, 1H), 3.96-3.92 (m, 1H), 3.25 (m, 1H), (2.99 (m, 2H); ¹³C NMR (100 MHz, D₂O) δ major diastereomer: 175.0, 137.1, 136.9, 129.3, 128.0, 127.9, 127.6, 126.1, 53.7, 53.6, 24.6; minor diastereomer: 175.4, 138.0, 136.7, 129.7, 127.9, 127.7, 127.4, 125.3, 58.5, 51.3, 24.2; HRMS found [M+H]⁺found [M+H]⁺ 250.0646, C₁₁H₁₂N₃O₂S requires 250.0645.

The tetrahydro-intermediate (III) (mixture of diasteromers) was dehydrogenated with IBX (1.5 equiv.) in DMSO at 45° C. for 6 hrs. DMSO was removed on the freeze-drier to yield a yellow crude residue which was first purified using column chromatography over silica using a gradient of 5:95-45:55 (MeOH:CHCl₃ saturated with ammonia) then by HPLC (gradient 0-70% acetonitrile/water/0.1% TFA) to yield the TFA salt of 4-(thiophen-3-yl)-3H-imidazo[4,5-c]pyridine-6-carboxylic acid. Isolated as the sole product as a white solid (75%). ¹H NMR (400 MHz, DMSO-d₆) δ 9.76 (dd, J=3.1, 1.2 Hz, 1H), 8.69 (s, 1H), 9.17 (dd, J=5.0, 1.2 Hz, 1H), 8.24 (s, 1H), 8.48 (dd, J=5.1, 3.1 Hz, 1H); ¹³C NMR (100 MHz, DMSO-d₆) δ 171.5, 151.2, 148.2, 145.2, 144.8, 144.5, 143.5, 133.3, 133.1, 131.1, 113.7; HRMS found [M+H]⁺ 246.0333, C₁₁H₈N₃O₂S requires 246.0332.

### Example 30

This example describes the two step synthesis of the 2-furan analog, (I where R^{d}=2-furan, R^{a}═R^{b}═R^{c}═H), in two steps starting from histamine instead of histidine. To a stirred solution of histamine (51.8 mg, 0.467 mmol.) in ethanol (10 mL) were added 2-furfural (44.8 mg, 0.467 mmol.) followed by potassium carbonate (774.2 mg, 5.60 mmol.) and 3 Å molecular sieves (724 mg). The reaction mixture was refluxed (80° C.) for 1 day after which it was filtered and the residue washed with methanol (20 mL). The filtrate was evaporated in vacuo and the brown residue subjected to column chromatography over silica using a gradient of 10:90-40:60 (MeOH:CHCl₃ saturated with ammonia) to yield the ageladine A analog as a brown gum (67.4 mg, 78%) after purification by column chromatography over silica using a gradient of 2:98-15:85 (MeOH:CHCl₃ saturated with ammonia). ¹H NMR (400 MHz, D₂O) δ 7.62 (s, 1H), 7.49-7.48 (m, 1H), 6.43-6.42 (m, 1H), 6.24-6.23 m, 1H), 3.17-3.11 (m, 1H), 3.05-2.99 (m, 1H), 2.72-2.68 (m, 2H); ¹³C NMR (100 MHz, D₂O) δ154.3, 143.5, 135.6, 130.1, 127.8, 111.0, 109.1, 50.4, 10.3, 22.2; HRMS found [M+H]⁺ 190.0976, C₁₀H₁₂N₃O requires 190.0975.

The tetrahydro-intermediate (III) (mixture of diasteromers) was dehydrogenated with IBX (1.5 equiv.) in DMSO at 45° C. for 7 hrs. DMSO was removed on the freeze-drier to yield a yellow crude residue which was first purified using column chromatography over silica using a gradient of 5:95-45:55 (MeOH:CHCl₃ saturated with ammonia) then by HPLC (gradient 0-70% acetonitrile/water/0.1% TFA) to yield the TFA salt of 4-(furfur-2-yl)-3H-imidazo[4,5-c]pyridine, isolated as a white solid (30%). ¹H NMR (400 MHz, DMSO-d₆) δ 8.86 (s, 1H), 8.44 (d, J=6.4 Hz, 1H), 8.26 (dd, J=1.7, 0.7 Hz, 1H), 8.03 (d, J=3.48 Hz, 1H), 7.94 (d, J=6.2 Hz, 1H), 6.97 (dd, J=1.8, 3.4 Hz, 1H); ¹³C NMR (100 MHz, DMSO-d₆) δ 149.1, 148.2, 145.9, 144.0, 135.9, 135.1, 134.7, 119.2, 114.1, 109.7; HRMS found [M+H]⁺ 186.0664, C₁₀H₈N₃O requires 186.0662.

### Example 31

This example describes the synthesis of pyridine analog of ageladine A, (I where R^{d}=2-pyridine, R^{a}═R^{b}═R^{c}═H), in a one-pot reaction. To a stirred solution of histamine (20.0 mg, 0.180 mmol.) in ethanol (2 mL) were added pyridine-2-carboxaldehyde (19.3 mg, 0.180 mmol.) followed by potassium carbonate (298.8 mg, 2.16 mmol.) and 3 Å molecular sieves (200 mg). The reaction mixture was refluxed (80° C.) in a sealed tube for 1 day after which 10% Pd/C (60 mg) and cyclopentene (120 μL) were added. The reaction mixture was allowed to reflux for 2 more days. The mixture was then filtered through a pad of celite, washed with methanol (3×30 mL) and toluene (2×25 mL). The combined filtrates were concentrated under reduced pressure and chromatographed over silica using a gradient of 2:98-15:85 (MeOH:CHCl₃ saturated with ammonia) to yield the analog (48%) in one pot as clear colourless crystals. ¹H NMR (400 MHz, CD₃OD) δ 9.52 (d, J=7.2 Hz, 1H), 8.98 (bs, 1H), 8.91 (s, 1H), 8.61 (d, J=5.9 Hz, 1H), 8.23 (t, J=7.7 Hz, 1H), 8.19 (d, J=5.9 Hz, 1H), 7.75 (bs, 1H); ¹³C NMR (100 MHz, CDCl₃) δ 150.1, 148.9, 147.4, 146.5, 140.0, 138.6, 135.9, 134.7, 127.0, 126.5, 111.99; HRMS found [M+H]⁺ 197.0824, C₁₁H₉N₄ requires 197.0822.

### Example 32

This example describes the synthesis of pyridine analog of ageladine A, (I where R^{d}=6-bromo-2-pyridine, R^{a}═COOH, R^{b}═R^{c}═H), in a one-pot reaction. To a stirred solution of histidine (83.4 mg, 0.538 mmol.) in ethanol (15 mL) were added 6-bromo-2-pyridine carboxaldehyde (100 mg, 0.538 mmol.) followed by potassium carbonate (892 mg, 6.45 mmol.) and 3 Å molecular sieves (834 mg). The reaction mixture was refluxed (80° C.) for 1 day and 19 hrs after which it was filtered and the residue washed with methanol (30 mL). The filtrate was evaporated in vacuo and the yellow residue purified by HPLC (gradient 0-70% acetonitrile/water/0.1% TFA) to yield the TFA salt of the analog (120 mg, 71%) as a yellowish solid. ¹H NMR (400 MHz, DMSO-d₆) δ 12.68 (bs, 1H), 8.76 (d, J=7.72 Hz, 1H), 8.68 (s, 1H), 8.48 (s, 1H), 8.05 (t, J=7.8 Hz, 1H), 7.83 (dd, J=7.8, 0.78 Hz, 1H); ¹³C NMR (150 MHz, DMSO-d₆ at −10° C.) δ 168.7, 158.3, 152.3, 149.6, 142.7, 141.8, 140.6, 132.4, 130.5, 122.9, 119.7; LRMS (ESI) m/z 319 (100%), 321 (98%); HRMS found [M+H⁺] 318.9828, 320.9807, C₁₂H₈N₄O₂Br⁷⁹ requires 318.9831, C₁₂H₈N₄O₂Br⁸¹ requires 320.9810

### Example 33

This example describes the synthesis of 4-(1-(4-fluorophenyl)-1H-pyrazol-4-yl) analog of ageladine A, (I where R^{d}=4-(1-(4-fluorophenyl)-1H-pyrazol-4-yl), R^{a}═COOH, R^{b}═R^{c}═H), in a one-pot reaction. To a stirred solution of histidine (83.4 mg, 0.538 mmol.) in ethanol (15 mL) were added 1-(4-fluorophenyl)-1H-pyrazole-4-carbaldehyde (102 mg, 0.538 mmol.) followed by potassium carbonate (892 mg, 6.45 mmol.) and 3 Å molecular sieves (834 mg). The reaction mixture was refluxed (80° C.) for 1 day and 19 hrs after which it was filtered and the residue washed with methanol (30 mL). The filtrate was evaporated in vacuo and the yellow residue purified by HPLC (gradient 0-70% acetonitrile/water/0.1% TFA) to yield the TFA salt of the analog as a pale yellow solid (52 mg, 51%). ¹ H NMR (400 MHz, DMSO-d₆) δ 9.50 (s, 1H), 8.89 (s, 1H), 8.70 (s, 1H), 8.22 (s, 1H), 8.02 (m, 2H), 7.45 (m, 2H); ¹³C NMR (150 MHz, DMSO-d₆) δ 167.0, 162.7, 160.3, 147.0, 142.2, 140.9, 140.8, 138.8, 137.0 (d, J=2.5 Hz), 129.2, 123.0, 121.7 (d, J=9.9 Hz), 117.4 (d, J=22.7 Hz), 108.8; HRMS found [M+H⁺] 324.0892, C₁₆H₁₁FN₅O₂ requires 324.0897.

### Example 34

A potentially non-toxic method of treating cancer is to inhibit angiogenesis. The human anti-angiogenic and therefore antiproliferative action of drugs can be assessed by many methods well known in the prior art. For example, culturing placental vessels in a fibrin gel and measuring the effect of different substances on the growth of micro-vessels from the severed ends of the vessel fragment into the surrounding fibrin gel can be used to assess the anti-angiogenic activity of these substances (Brown, K. J.; Maynes, S. F.; Bezos, A.; Maguire, D. J.; Ford, M. D.; Parish, C. R. Lab. Invest. 1996, 75, 539). To assess the anti-angiogenic activity the ability of ageladine A analogs to inhibit the outgrowth of new vessels from isolated rat aorta fragments was measured. Briefly, thoracic aortas were excized from 3-9 month-old female Fischer rats, cross-sectioned at 1 mm intervals and embedded individually in 0.5 ml of a fibrin gel containing 5 μg/ml of aprotinin (to prevent fibrinolysis) in each well of a 48-well culture plate. Immediately after vessel embedding, 0.5 ml/well of medium M199, supplemented with 20% foetal calf serum, 0.1% E-aminocaproic acid, 1% L-glutamine and antibiotics, was added. Each test compound (10 mg/ml, dissolved in DMSO) was added to the medium (maximum final concentration of test compound 100 μg/ml) and each treatment was performed in six wells. Control cultures received medium containing 0.1% DMSO but without any test compound. The anti-angiogenic compound, PI-88 (100 μg/ml), was included as a positive control (Parish, C. R.; Freeman, C.; Brown, K. J.; Francis, D. J.; Cowden, W. B. Cancer Res. 1999, 59, 3433). Vessels were cultured at 37° C. in 5% CO₂ in air for 5 days and the medium, with or without test compound, was changed on day 4. Vessel growth was quantified manually under 40× magnification on day 5, with growth being estimated as the percentage of the field (×40) around the vessel fragment that was occupied by vessel outgrowths. By counting the number of vessels formed after 5 days at three to six different concentrations (in triplicate) it is possible to determine an IC₅₀ values for each compound compared to a positive and negative (solvent) control. The percentage inhibition (0-100%) is plotted against compound concentration and a 3-parameter sigmoidal (dose-response) curve fitted (Graphpad Prism v 4) by non-linear regression. The results are presented in Table 1. Surprisingly, the 2-pyridine analogs all showed good activity with 4-(pyrid-2-yl)-1H-imidazo[4,5-c]pyridine, (I where R^{b}═H, R^{d}=2-pyridyl, R^{a}═R^{c}═H) showing much greater activity than the natural product. Even more surprisingly, moving the nitrogen around the ring by just one position (4-(pyrid-3-yl)-1H-imidazo[4,5-c]pyridin-2-amine, (I where R^{b}═NH₂, R^{d}=3-pyridine, R^{a}═R^{c}═H) abolished all activity in this assay.

### Example 35

Percentage kinase inhibition at a set concentration was performed by methods well known in the prior art (Fabian, M. A. et al. Nat. Biotechnol. 2005, 23, 329; Karaman et al. (2008) Nat. Biotechnol. 2005, 26, 127). Briefly, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. Streptavidin-coated magnetic beads were treated with biotinylated small molecule ligands (specific for one kinase) for 30 minutes at room temperature to generate affinity resins for kinase assays. The liganded beads were blocked with excess biotin and washed with blocking buffer (SeaBlock (Pierce); 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17× PBS, 0.05% Tween 20, 6 mM DTT). Test compounds were prepared as 40× stocks in 100% DMSO and directly diluted into the assay. All reactions were performed in polypropylene 384-well plates in a final volume of 0.04 mL. The assay plates were incubated at room temperature with shaking for 1 hour and the affinity beads were washed with wash buffer (1× PBS, 0.05% Tween 20). The beads were then resuspended in elution buffer (1× PBS, 0.05% Tween 20, 0.5 μm non-biotinylated affinity ligand) and incubated at room temperature with shaking for 30 minutes. The kinase concentration in the eluates was measured by qPCR.

The compounds were screened at 1 μM (and 10 μM for ageladine A) and binding interactions are reported as % Ctrl=(test compound signal−positive control signal)/negative control signal−positive control signal) such that number less than 100 indicate inhibition of that kinase. Results for ageladine A and two analogs are given in Table 2.

### Example 36

Kinase inhibition studies were performed by methods well known in the prior art (Fabian, M. A. et al. Nat. Biotechnol. 2005, 23, 329). Briefly, kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce); 1% BSA, 0.05% Tween 20, 1 mM DTT) to remove unbound ligand and to reduce non-specific binding. Binding reactions were assembled by combining kinases, liganded affinity beads, and test compounds (I) in 1× binding buffer (20% SeaBlock, 0.17× PBS, 0.05% Tween 20, 6 mM DTT). All reactions were performed in polystyrene 96-well plates in a final volume of 0.135 ml. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μm non-biotinylated affinity ligand) and incubated at room temperature with shaking for 30 minutes. The kinase concentration in the eluates was measured by qPCR.

An 11-point 3-fold serial dilution of each test compound (from 30 μM to 0.5 nM) was prepared in 100% DMSO at 100× final test concentration and subsequently diluted to 1× in the assay (final DMSO concentration=1%). Binding constants (K_{D}S) were calculated with a standard dose-response curve using a 5-point sigmoidal (Hill) equation. Curves were fitted using a non-linear least squares approach (GraphPad Prism v 4) and presented in Table 3.

### Example 37

Anticancer activity is commonly associated with the inhibition of kinase enzymes. To determine if the novel ageladine A analogs described here could be used as anti-cancer drugs, kinase activities can be assayed in Buffer A or C, at 30° C., at a final ATP concentration of 15 μM. Blank values are subtracted and activities expressed as percent of the maximal activity, i.e. in the absence of inhibitors (Table 4). Controls were performed with appropriate dilutions of DMSO, which was used to dissolve all test compounds.

CDK1/cyclin B (native M phase starfish oocytes), and CDK5/p25 (recombinant human) can be prepared according to the prior art (Meijer, L.; Thunnissen, A. M. W. H.; White, A. W.; Gamier, M.; Nikolic, M., et al. Chem. Biol. 2000, 7, 51-63). Kinase activity can be assayed in buffer C, with 1 mg histone H1/mL, in the presence of 15 μM [γ-³²P] ATP (3,000 Ci/mmol, 10 mCi/mL) in a final volume of 30 μL. After 30 min. incubation at 30° C., 25 μL aliquots of supernatant are spotted onto 2.5×3 cm pieces of Whatman P81 phosphocellulose paper and, 20 s later, the filters are washed five times (for at least 5 min. each time) in a solution of 10 mL phosphoric acid/L of water. The wet filters are then counted in the presence of 1 mL ACS (Amersham) scintillation fluid.

CK1 (porcine brain, native) can be assayed as described for CDK1 but using the CK1-specific peptide substrate RRKHAAIGpSAYSITA (Reinhardt, J.; Ferandin, Y.; Meijer, L. Prot. Expr. Purif. 2007, 54, 101-9), which can be obtained from Millegen (Labege, France). DYRK1A (rat, recombinant, expressed in E. coli as a GST fusion protein) can be purified by affinity chromatography on glutathione-agarose and assayed as described for CDK1/cyclin B using myelin basic protein (1 mg/mL) as a substrate. Pim1 (human recombinant) can be assayed with histone H1 as described for CDK1 and CDK5.

### Example 38

Anti-angiogenic activity is often associated with inhibition of matrix metalloprotease (MMP) enzymes. To assay the MMP inhibition of the analogs described here, recombinant human MMP-2 and MT1-MMP are prepared following the literature procedures. The fluorescent substrate; MOCAc-Pro-Leu-Gly-Leu-A₂pr(Dnp)-Ala-Arg-NH₂ can be purchased from Peptide Institute Inc., Osaka. Inhibition assays were carried out by a modified procedure of Knight et al. (Knight, C. G.; Willenbrock, F.; Murphy, G. FEBS Lett. 1992, 296, 263-6). Test samples (2 μL), including positive (BB-94; IC₅₀ 0.29, 1.45 μg/mL for MMP-2 and MT1-MMP respectively) and negative (1% DMSO) controls, are added to wells of 96-well microtiter plates, each of which contains 100 μL of TNC buffer (50 mM Tris-HCl pH 7.5, 150 mM NaCl, 10 mM CaCl₂, 0.02% NaN₃ as a preservative and 0.05% Brij-35 detergent). Aliquots (50 μL) of enzyme solution (5 ng/mL for MMP-2 and 40 ng/mL for MT1-MMP) are added to this solution, and pre-incubated at 37° C. for 10 min. After pre-incubation, 50 μL of substrate solution (10 μM) is added to the mixture to begin the reaction. Fluorescence values can be measured at an excitation of 328 nm and an emission of 393 nm after incubation at 37° C. for 3 h. Reference samples with all components except the enzyme are used as blanks and subtracted from all assay readings (Table 5). Contrary to the teachings of the prior art, the anti-angiogenic activity of ageladine A does not seem to be related to inhibition of MMP enzymes (Fujita, M.; Nakao, Y.; Matsunaga, S.; Seiki, M.; Itoh, Y., et al. J. Am. Chem. Soc. 2003, 125, 15700-1). This unexpected result may suggest that these compounds act on angiogenesis by a new mechanism.

TABLE 1a
Anti-angiogenic activity of ageladine A and 10 analogs (where R^{b} = NH₂,
R^{a} = [symbol] R^{c} = H) expressed as a concentration (μg/mL) that inhibits 50%
of the new blood vessel growth in the assay. A value of >100 indicates
that there is no inhibition under the assay conditions.

| | Anti- |
|---|---|
| | Angiogenesis |
| R^{d} = IC₅₀ μg/mL | Activity |
| Ageladine A | 25 |
| 2-pyrrole | >100 |
| 2-furan | >100 |
| 2-thiophene | 10 |
| 3-furan | >100 |
| 3-thiophene | >100 |
| 2-pyridine | 8 |
| 3-pyridine | >100 |
| 3-quinoline | 75 |
| phenyl | >100 |
| 3-indole | >100 |

[symbol]

indicates data missing or illegible when filed

TABLE 1b
Anti-angiogenic activity of ageladine A and 5 analogs (where R^{b} =
H, R^{a} = COOH, R^{c} = H) expressed as a concentration that
inhibits 50% of the new blood vessel growth in the assay. A value
of >100 indicates that there is no inhibition under the assay conditions.

| | Anti- |
|---|---|
| | Angiogenesis |
| R^{d} = IC₅₀ μg/mL | Activity |
| Ageladine A | 25 |
| 2-pyridine | 31 |
| 6-bromo-2-pyr | 16 |
| 3-pyridine | >100 |
| 3-furan | >100 |
| 3-thiophene | >100 |

TABLE 1c
Anti-angiogenic activity of ageladine A and 2 analogs (where R^{b} = H,
R^{a} = H, R^{c} = H) expressed as a concentration that inhibits 50% of
the new blood vessel growth in the assay. A value of >100 indicates
that there is no inhibition under the assay conditions.

| | Anti- |
|---|---|
| | Angiogenesis |
| R^{d} = IC₅₀ μg/mL | Activity |
| Ageladine A | 25 |
| 2-pyridine | 15 |
| 3-thiophene | >100 |

TABLE 2
Kinase inhibition results expressed as % inhibition for ageladine A and two analogs (I where R^{b} = NH₂, R^{a} = R^{c} = H).

| | R^{d} = | | | |
|---|---|---|---|---|
| | 1 μM | 1 μM | 1 μM | 10 μM |
| Kinase | 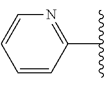 | 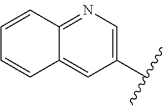 | 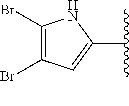 | 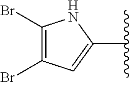 |
| MKNK2 | 86 | 100 | 2.5 | 69 |
| RPS6KA1(Kin.Dom.2-C-terminal) | 33 | 92 | 20 | 22 |
| TYK2(JH2domain-pseudokinase) | 41 | 25 | 23 | 2 |
| DYRK2 | 41 | 56 | 35 | 4.4 |
| DYRK1A | 36 | 51 | 38 | 4.7 |
| RPS6KA2(Kin.Dom.2-C-terminal) | 68 | 100 | 42 | 25 |
| GAK | 54 | 91 | 45 | 19 |
| CLK4 | 60 | 65 | 51 | 11 |
| RPS6KA6(Kin.Dom.2-C-terminal) | 49 | 100 | 51 | 34 |
| CDKL5 | 50 | 54 | 54 | 40 |
| PRKCE | 20 | 100 | 54 | 100 |
| IRAK1 | 52 | 89 | 55 | 35 |
| YSK4 | 16 | 82 | 56 | 5.1 |
| PIM2 | 100 | 100 | 56 | 21 |
| ULK1 | 33 | 91 | 62 | 20 |
| ROCK2 | 45 | 100 | 65 | 38 |
| PIK4CB | 88 | 37 | 66 | 50 |
| MAP3K2 | 54 | 81 | 73 | 70 |
| DRAK1 | 56 | 100 | 78 | 86 |
| CDK7 | 30 | 100 | 79 | 66 |
| GRK1 | 49 | 86 | 80 | 85 |
| ZAP70 | 54 | 94 | 81 | 65 |
| PLK4 | 55 | 100 | 81 | 65 |
| ULK3 | 51 | 87 | 82 | 81 |
| TBK1 | 50 | 79 | 84 | 78 |
| IRAK3 | 88 | 56 | 85 | 51 |
| SBK1 | 53 | 100 | 87 | 77 |
| SgK085 | 43 | 92 | 89 | 90 |
| AAK1 | 27 | 83 | 90 | 83 |
| CAMKK2 | 35 | 100 | 90 | 83 |
| CAMKK1 | 19 | 100 | 92 | 100 |
| YANK2 | 89 | 50 | 93 | 87 |
| PIK3C2G | 66 | 30 | 99 | 80 |
| BMPR1B | 85 | 0 | 100 | 78 |
| MAP3K15 | 40 | 94 | 100 | 78 |
| CAMK2G | 20 | 100 | 100 | 78 |
| CAMK2D | 41 | 100 | 100 | 82 |
| CAMK1G | 43 | 100 | 100 | 96 |
| CAMK2A | 100 | 100 | 100 | 97 |
| CAMK4 | 100 | 100 | 100 | 97 |

A value of 100 indicates that there was no inhibition at the tested concentration.
A value of <50 indicates that inhibition is in the nM range for columns 1-3.

In Table 2, the kinases that were specific to Ageladine A are shown in normal type, 2-pyridine analog are italicized and quinoline analog are underlined. Specificity is important in kinase-acting drugs as they need to affect only one (or a few) kinases strongly and not affect others. Commonly, kinase inhibitors have low specificity, inhibiting all or many kinases. These may be less effective as drugs by causing multiple side effects. The three compounds of Table 2 appear to affect only a few kinases, but surprisingly, they affect different groups.

TABLE 3
Kinase inhibition results for two analogs (I where R^{b} = NH₂, R^{a} = R^{c} = H).
A blank indicates that this kinase was not tested and >30 indicates that
there was no inhibition even at the highest concentration tested.

| R^{d} = | 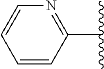 | 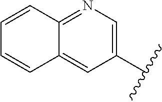 |
|---|---|---|
| Kinase | K_{D} (μM) | K_{D} (μM) |
| AAK1 | 1.5 | |
| BMPR1B | | 7.2 |
| CAMK2G | >30 | |
| CAMKK1 | >30 | |
| CAMKK2 | >30 | |
| CDK7 | 7.5 | |
| CDKL5 | | >30 |
| CLK2 | | 1.2 |
| DYRK1A | 0.29 | 0.30 |
| DYRK2 | | 0.97 |
| IRAK3 | | 8.9 |
| PIK3C2G | | 160 |
| PIK4CB | | 0.35 |
| PRKCE | >30 | |
| RSK1 | 0.53 | |
| TYK2 | | 0.29 |
| ULK1 | 24 | |
| YANK2 | | >30 |
| YSK4 | 0.22 | |

TABLE 4
Effect (IC₅₀ values in μM) of ageladine A (I where R^{b} = NH₂, R^{a} = R^{c} = H) and
analogs on the activity of five protein kinases.^{a}

| | | CDK1/ | CDK5/ | | | |
|---|---|---|---|---|---|---|
| R^{d} = | Compound | cyclin B | p25 | CK1 | Dyrk1A | Pim 1 |
| 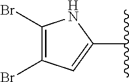 | ageladine A (1) | — | — | 50 | 30 | 10 |
| 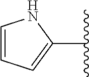 | 2 | — | 70 | — | — | — |
| 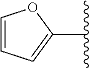 | 3 | — | 34 | — | — | — |
| 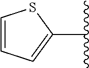 | 4 | — | 43 | — | 80 | — |
| 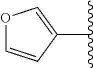 | 5 | — | — | — | — | — |
| 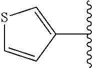 | 6 | 40 | 11 | — | 11 | — |
| 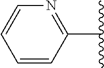 | 7 | 9 | 6 | 10 | 5 | — |
| 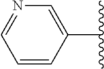 | 8 | — | 60 | — | 10 | — |
| 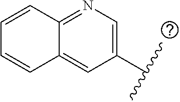 | 9 | — | — | — | 7 | 40 |
| 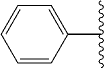 | 10 | — | 40 | — | 40 | — |
| 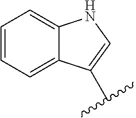 | 12 | — | — | — | 40 | 50 |

^{a}Compounds were tested at various concentrations up to 100 μM and IC₅₀ values (μM) were calculated from the dose-response curves.
—: inactive at 100 μM

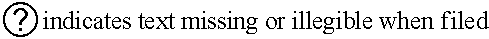

TABLE 5
MMP inhibition (IC₅₀) results for ageladine A and analogs
(I where R^{b} = NH₂, R^{a} = R^{c} = H).

| | | MMP-2 | MT1-MMP |
|---|---|---|---|
| R^{d} = | compound | (μg/mL) | (μg/mL) |
| 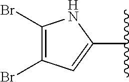 | ageladine A (1) | 1.7 | 0.2 |
| 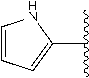 | 2 | 10 | 1.6 |
| 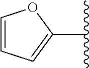 | 3 | 12.4 | 3.2 |
| 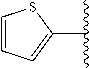 | 4 | 3.0 | 0.57 |
| 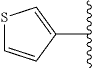 | 6 | 19 | 6.4 |
| 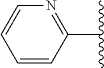 | 7 | 22% ^{a} | 6% ^{a} |
| 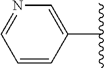 | 8 | 5% ^{a} | — |
| 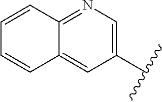 | 9 | 11% ^{a} | 9% ^{a} |
| 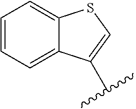 | 11 | 21% ^{a} | 17% ^{a} |
| 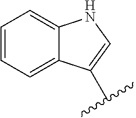 | 12 | 31% ^{a} | 2.8 |

^{a} percentage inhibition at 20 μg/mL

1. A one-pot process for synthesizing a compound of structure I, or a tautomer thereof, said process comprising:
condensing a compound of structure II, or a tautomer thereof, and an aldehyde of structure R^{d}CHO to form a condensation product; and
oxidizing the condensation product to produce the compound of structure I or tautomer thereof; 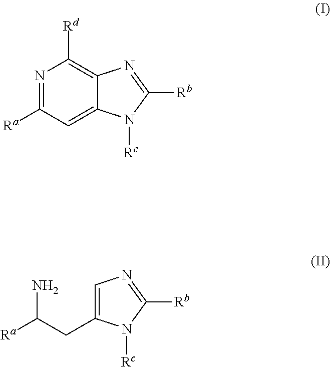
wherein:
R^{a} is R¹, COOR¹ or CONHR¹,
—R^{b} is H, —NR¹R², —OR¹, —SR¹, —SO₂R¹, or —NR³, where R³ comprises a hydrocarbon chain which, together with the nitrogen to which it is attached, forms a ring structure,
R^{c} is R¹,
R^{d} is an optionally substituted aromatic or heteroaromatic group,
R¹ and R² are, independently, H, alkyl, aryl or acyl, and are, except if they are H, optionally substituted, wherein each R¹ is independently as defined above.

2.-4. (canceled)

5. The process of claim 1 wherein the compound of structure II is 2-aminohistamine and the aldehyde is N-Boc-4,5-dibromopyrrole-2-carboxaldehyde, whereby the compound of structure I is ageladine A.

6. The process of claim 1 wherein the step of oxidizing comprises heating the crude reaction product in the presence of a reagent selected from the group consisting of palladium on charcoal catalyst, a quinone, sulfur and iodoxybenzoic acid.

7. The process of claim 6 wherein the reagent is palladium on charcoal catalyst and the heating comprises refluxing in ethanol.

8. The process of claim 6 wherein the reagent is chloranil or DDQ and the heating comprises refluxing in a halogenated solvent.

9. The process of claim 1 wherein the step of condensing comprises reacting said compound of structure II with said aldehyde in the presence of a base, said base being different to the compound of structure II.

10. The process of claim 9 wherein the base is a tertiary amine or an alkali metal hydroxide or alkali metal carbonate.

11. The process of claim 1 wherein R¹ and R² are substituted with one or more hydroxy, halo or amino groups.

12. The process of claim 1 additionally comprising isolating the compound of structure I or tautomer thereof.

13.-14. (canceled)

15. A compound of structure I for treating hyperproliferative diseases and/or other diseases associated with angiogenesis 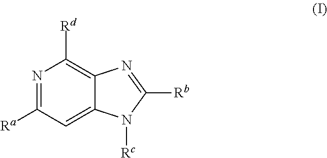
wherein:
R^{a} is R¹, COOR¹ or CONHR¹,
—R^{b} is H, —NR¹R², —OR¹, —SR¹, —SO₂R¹, or —NR³, where R³ comprises a hydrocarbon chain which, together with the nitrogen to which it is attached, forms a ring structure,
R^{c} is R¹,
R^{d} is an optionally substituted aromatic or heteroaromatic group,
R¹ and R² are, independently, H, alkyl, aryl or acyl, and are, except if they are H, optionally substituted, wherein each R¹ is independently as defined above;
or a tautomer, pharmaceutically acceptable salt or ester thereof,
wherein R^{a}, R^{b}, R^{c} and R^{d} are not any of the combinations shown in the table below:

| R^{a} | R^{b} | R^{c} | R^{d} |
|---|---|---|---|
| H | NH₂ | H | 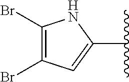 |
| H | NH₂ | H | 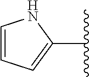 |
| H | NH₂ | H | 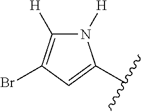 |
| H | NH₂ | H | 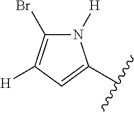 |
| H | NH₂ | H | 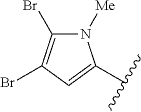 |
| H | NH₂ | H | 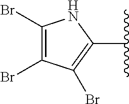 |
| H | NHMe | H | 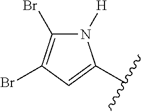 |
| H | NH₂ | Me | 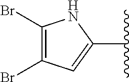 |
| H | NHMe | Me | 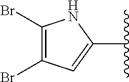 |
| H | NMe₂ | H | 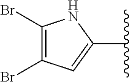 |
| H | H | H | 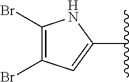 |
| H | NH₂ | Bom | 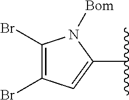 |
| H | NH₂ | Bom | 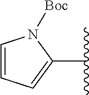 |
| H | NH₂ | Bom | 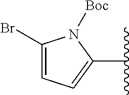 |
| H | NH₂ | Bom | 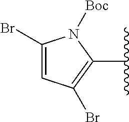 |
| H | NH₂ | H | 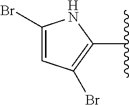 |
| H | N₃ | Bom | 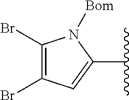 |
| H | SO₂Me | Bom | 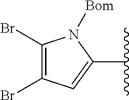 |
| H | SOMe | Bom | 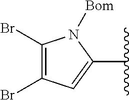 |
| H | SMe | Bom | 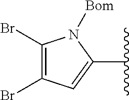 |
| H | NHBoc | H | 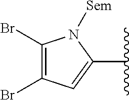 |
| H | NMeBoc | H | 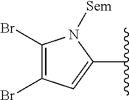 |
| H | NMeBoc | Me | 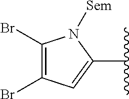 |
| H | H | H | 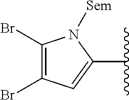 |
| H | H | Me | 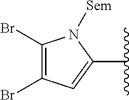 |
| H | Br | H | 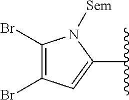 |
| H | NMe₂ | H | 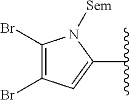 |
| H | NHMe | Me | 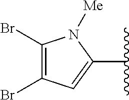 |
| CO₂Me | H | H | Ph |
| H | H | 4-hydroxy-n-butyl | 2,4-dichlorophenyl |
| H | [p-(trimethyl-silyl)ethoxy]methyl | H | Ph |
| H | SMe | H | Ph |
| H | H | H | Ph |
| H | SMe | PMB | 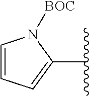 |
| H | SMe | PMB | 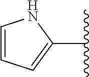 |
| H | S(O)Me | PMB | 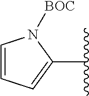 |
| H | H | BOM | 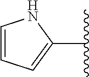 |

16. The compound of claim 15 wherein R¹ and/or R² are substituted with one or more hydroxy, halo or amino groups.

17. The compound of claim 11 or claim 15, wherein R^{a} and R^{c} are both H, R^{b} is NH₂ and R^{d} is selected from the group consisting of furan-2-yl, thiophen-2-yl, furan-3-yl, thiophen-3-yl, pyridin-2-yl, pyridin-3-yl, quinolin-3-yl, phenyl, benzothiophen-3-yl and indol-3-yl, or wherein R^{a} is COOH, R^{b} and R^{c} are both H and R^{d} is thiophen-2-yl, pyridin-2-yl or 6-bromopyridin-2-yl, or such that R^{a}, R^{b} and R^{c} are all H and R^{d} is pyridin-2-yl.

18. The compound of claim 17 wherein R^{a} and R^{c} are both H, R^{b} is NH₂ and R^{d} is thiophen-2-yl, pyridine-2-yl or quinolin-3-yl, or wherein R^{a} is COOH, R^{b} and R^{c} are both H and R^{d} is thiophen-2-yl, pyridin-2-yl or 6-bromopyridin-2-yl, or such that R^{a}, R^{b} and R^{c} are all H and R^{d} is pyridin-2-yl said compound having anti-angiogenic activity whereby at least 50% of new blood vessel growth is inhibited at a concentration of said compound of <100 μg/mL.

19. The compound of claim 17 wherein R^{a} and R^{c} are both H, R^{b} is NH₂ and R^{d} is furan-2-yl, thiophen-2-yl, furan-3-yl, thiophen-3-yl, pyridin-2-yl, pyridin-3-yl, quinolin-3-yl, phenyl or indol-3-yl, said compound having kinase inhibitor activity of IC₅₀<100 μM.

20. The compound of claim 17 wherein R^{a} and R^{c} are both H, R^{b} is NH₂ and R^{d} is furan-2-yl, thiophen-2-yl, thiophen-3-yl, pyridin-2-yl, pyridin-3-yl, quinolin-3-yl, benzothiophen-3-yl or indol-3-yl, said compound having MMP inhibitor activity.

21. A pharmaceutical composition for treating hyperproliferative diseases and/or other diseases associated with angiogenesis, said composition comprising a compound according to claim 15, or a tautomer thereof, or a pharmaceutically acceptable salt or ester thereof or a combination of any two or more of said compounds, tautomers, salts and esters, together with one or more pharmaceutically acceptable carriers and/or adjuvants.

22.-24. (canceled)

25. A method of treating a hyperproliferative disorder or an angiogenic disease in a patient comprising administering to said patient a therapeutically effective amount of a compound according to claim 15, or a tautomer thereof, or a pharmaceutically acceptable salt or ester thereof or a combinations of any two or more of said compound, salt and ester, or of a composition according to claim 21, wherein said compound is effective against said disorder or disease.

26. The method of claim 25 wherein R^{a} and R^{c} are both H, R^{b} is NH₂ and R^{d} is selected from the group consisting of furan-2-yl, thiophen-2-yl, furan-3-yl, thiophen-3-yl, pyridin-2-yl, pyridin-3-yl, quinolin-3-yl, phenyl, benzothiophen-3-yl and indol-3-yl, or wherein R^{a} is COOH, R^{b} and R^{c} are both H and R^{d} is thiophen-2-yl, pyridin-2-yl or 6-bromopyridin-2-yl, or such that R^{a}, R^{b} and R^{c} are H and R^{d} is pyridin-2-yl.

27. The method of claim 25 being a method of treating a cancer.

28. The method of claim 25 being a method of treating age related macular degeneration.
